(19) United States
(12) Patent Application Publication
(10) Pub. No.: US 2002/0009488 A1
(43) Pub. Date: Jan. 24, 2002

(54) Tissue entrapment

(75) Inventors: Gillian E. Francis, London (GB); Derek Fisher, London (GB)

Correspondence Address: Nixon & Vanderhye P.C., 8th Floor, 1100 N. Glebe Rd, Arlington, VA, 22201-4714, US

(73) Assignee: POLYMASC PHARMACEUTICALS PLC

(21) Appl. No.: 09/735,663

(22) Filed: Dec. 14, 2000

Related U.S. Application Data
(62) Division of application No. 09/327,172, filed on Jun. 7, 1999.
Application No. 09/327,172 is a continuation of application No. 08/765,349, filed on Dec. 31, 1996, now abandoned.

(30) Foreign Application Priority Data
May 3, 1995 (GB) 9509016.3
Jun. 7, 1995 (WO) PCT/GB95/01311

(51) Int. Cl. A61K 9/127

(52) U.S. Cl. 424/450

(57) ABSTRACT

Delivery of diagnostic and therapeutic agents to skin or solid tumours is improved by optimisation of the lipid containing macromolecular structures (eg liposomes) encapsulating the agents and the type and amount of hydrophilic moieties bound to the exterior of the macromolecular structures as well as the relative proportions of the various lipids or other hydrophobic entities forming the macromolecular structures.

The present invention relates to lipid-based compositions for delivering diagnostic and therapeutic materials to tumours and skin and to the use of such compositions in medicine.

A variety of disclosures have been made previously in relation to liposomal formulations bearing polyethylene glycol (PEG) moieties or associated with polyethylene glycols and similar polymers, where treatment of the liposomes is intended to provide stabilisation of the liposomes so as to enhance circulation lifetime, or to modulate the clearance of the liposomes from the circulation, or otherwise to facilitate delivery of the liposomes and the entrapped diagnostic or therapeutic agents to tumours. These disclosures have tacitly or explicitly relied upon what will be referred to hereafter as a “push” mechanism for enhancing the tumour uptake of the diagnostic or therapeutic agent. Put simply, in accordance with the “push” mechanism, the more liposomes there are in the bloodstream and the longer they remain in the bloodstream, the more chance there is for the “payload” (the entrapped therapeutic or diagnostic agent or agents) to be delivered to the tumour.

The present inventors have arrived at an alternative mechanism for enhancing the delivery of a payload in a lipid-based composition to a solid tumour; the mechanism will be referred to hereafter as the “trap” mechanism. In simple terms, the “trap” mechanism operates by specifically reducing loss from the tumour of the lipid-based composition and thus retaining greater quantities of the delivered payload within the tumour.

The two mechanisms will be discussed in further detail below:

### The “Push” Mechanism

PCT/US 90/06211, (Liposome Technology Inc) in describing a tumour localisation method states at section IV, line 20:

“as detailed above, the liposomes of the invention are effective to localise specifically in a solid tumour region by virtue of the extended lifetime of the liposomes in the blood stream and a liposome size which allows both extravasation into tumours, a relatively high drug carrying capacity and minimal leakage of the entrapped drug during the time required for the liposomes to distribute to and enter the tumour (the first 24 to 48 hours following injection)”. This view of how tumour localisation takes place appears justified in the light of the data presented in the same patent application. Where there is an extended lifetime of a material in the blood stream and that material is capable of extravasation into a tumour site, there will automatically be an increased delivery of material to the tumour (in terms of the absolute amount delivered per unit time) because the extravasation of liposomes, which is unlikely to be a saturable process in view of current opinions on its mechanism, will increase pari passu with the blood concentration. In fact, since leaky tumour vasculature is well known, this implies that any liposome capable of leaking out will extravasate at an absolute rate dependent on the blood concentration. This localisation method is simply a matter of utilising the enhanced circulation time to “push” more material into the tumour by making more liposomes and hence more payload available per unit time.

Table 10 of PCT/US 90/06211 bears out this interpretation. If one uses Table 10 to calculate tumour to blood ratios, the conventional liposome control showed tumour to blood ratios of 0.1:1 at 2 h, 0.5:1 at 24 h and 1.4:1 at 48 h. This indicates that the control liposomes enter the tumour slowly (with respect to the blood clearance time), such that there is more material in the blood than the tumour at the first two time points. The persistence of material after the blood level has fallen indicates that the clearance rate for the material in the tumour is slower than that from the blood. Note that the blood clearance rate is a composite of the tissue distribution rate and the rate of elimination from the circulation by the usual excretion and destruction/metabolic process; and that the former predominates at early time points and the latter at late time points. In the example given in Table 10 of a PEGylated liposome (DSPC=10: Chol=3: PEG-PE=1 mole ratios), the tumour to blood ratios are 0.1:1 at 2 h, 0.3:1 at 24 h and 0.6:1 at 48 h, i.e. at the later two time points the ratios have fallen with respect to the un-PEGylated control. This reduction in tumour to blood ratios with respect to control at late time points is what is expected in most situations when entry into one or more of the body's liposome eliminating organs is reduced by polymer derivatisation, leading to an enhanced circulation time (see discussion on tumour to blood ratios below).

A further example in the same patent application (with no comparison to an unPEGylated control) also showed very low tumour to blood ratios (based on comparison of the doxorubicin contents of the liposome):

4 h 3.8:232=0.016 (ratio of μg doxorubicin/ml)

24 h 23:118=0.19 (ratio of μg doxorubicin/ml)

48 h 29.1:84=0.35 (ratio of μg doxorubicin/ml)

Thus these data support the interpretation of the underlying principle that in order to push more compound into the tumour it is necessary to ensure that the liposomes have the maxim retention time in the blood, and that they are small enough to traverse the blood/tumour barrier. It is implicit in this view that the longer the blood circulation time, the greater the amount of liposomes delivered to the tumour. The optimum formulation of these polymer coated liposomes is therefore to be achieved by maximising the retention time in the circulation. In order to do this, a combination of cholesterol-related and other lipid composition-related improvements in half life were further improved by PEGylation. Claim 9 of the same patent application emphasises the degree of increase in plasma half life to be achieved (“several times greater” than that of liposomes in the absence of derivitisation).

Several other publications disclose polymer-coated liposomes and discuss their tumour localising properties. In all these cases, where the data allow their calculation or the values are given, tumour to blood ratios are lower for the polymer-derivatised liposomes than the control non-derivatised liposomes and are less than 1 during some or all of the period between 24 and 48 h.

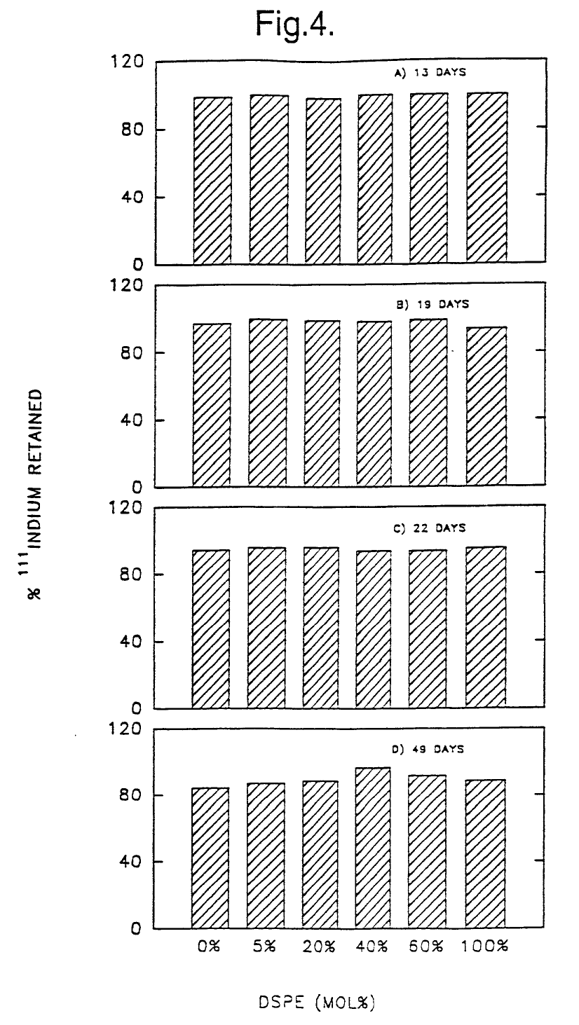

For example, FIG. 4 of Papahadjopoulous et al (PNAS, 88:11460-11464,(1991)) contains the same data that appears in PCT/US90/06211 with the addition of the uptake of doxorubicin into ascites located tumour cells:

PEGylated PEG-DSPE (0.2):ESPC (2):Chol (1); [PEG-DSP=6.25 mol % of lipids] Liposomes, (no unPEGylated control):

tumour to blood

| | | |
|---|---|---|
| 4 h | <1:232 | = <0.004 |
| 24 h | 0.5:118 | = 0.0042 |
| 48 h | 2:84 | = 0.024 |

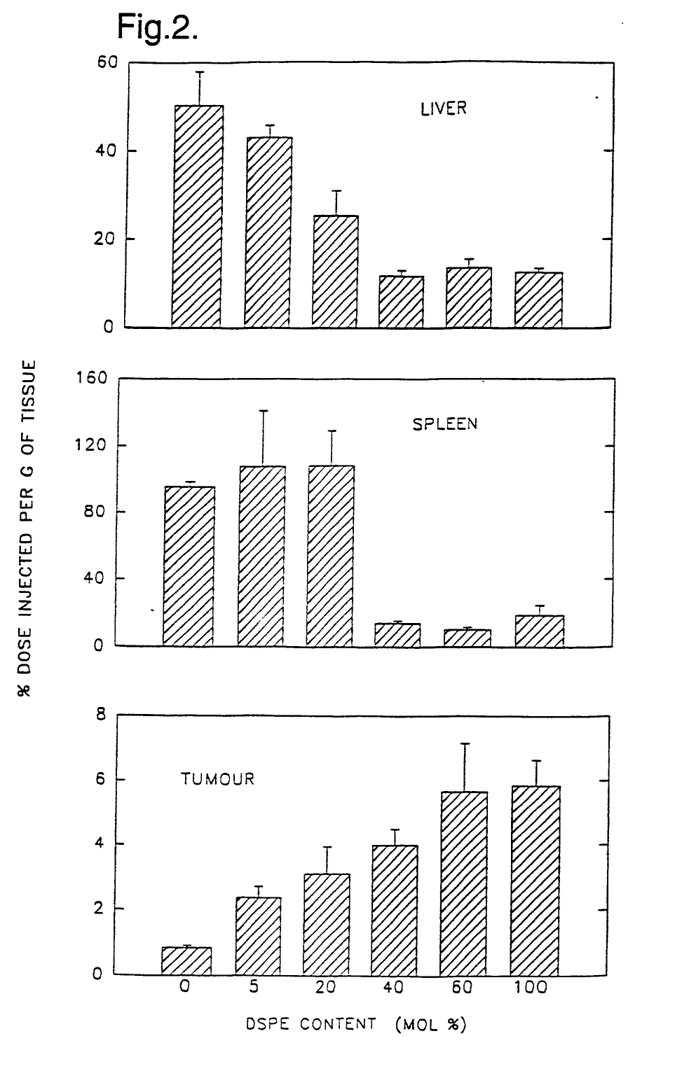

In addition, FIG. 2, of Hwang et al (Cancer Res., 52:6774-6781 (1992)) compares PEGylated and unmodified counterparts and shows Tumour to blood ratios at 48 h: for PEG-DSPE (0.2):DSPC(2):Chol(1) [PEG-DSPE=6.25 mol %, of lipids] vs DSPC(2):Chol(1)

tumour to blood

| | |
|---|---|
| 3:5 = 0.6 | (PEGlyated) |
| 2:1 = 2 | (UnPEGylated) |

### The “Trap” Mechanism

It should be noted that high tumour to blood concentration ratios (i.e. several fold above 1) are very desirable in many settings. Examples include tumour imaging of vascular organs, drug and radionuclide delivery. However, the present inventors have appreciated that achieving increased tumour localisation at the expense of reducing the tumour to blood ratios is undesirable. Experiments conducted by the present inventors with PEGylated liposomes have surprisingly revealed the possibility of achieving yet greater enhancement of tumour uptake by an alternative optimisation strategy which avoids reducing the tumour to blood ratio.

The basis of this invention is an examination of the factors influencing the tumour to blood ratio. Without wishing to be bound by their theory, the present inventors believe that enhancement of tumour uptake of diagnostic and therapeutic agents, delivered as the payloads in lipid-based structures bearing hydrophilic moieties, is achieved by influencing the rates of destruction by or loss of the lipid-based structures from the tumour, such that the payload material becomes trapped within the tumour. The inventors have shown that the skin is an organ which behaves in a similar fashion to solid tumours. Specifically, factors that change tumour to blood ratios in the optimisation procedures of the present invention were noted to have a similar impact on skin to blood ratios. This was not observed with other organs.

Although discussed below with reference to PEGylated liposomes, the principles for enhancing tumour and skin uptake can be extended to other PEGylated lipid-based structures and to lipid-based structures bearing hydrophilic polymer moieties other than PEG moieties. Also, for clarity, the optimisation of uptake is primarily discussed below with reference to tumours; nevertheless the principles are equally applicable to uptake by skin.

When the biodistribution of liposomes is altered by reducing their uptake by an eliminating tissue (i.e. an organ or cell type which destroys liposomes) the circulation half life is, inevitably, extended and more liposomally encapsulated material is delivered to the tumour. Under these circumstances the tumour to blood ratio of a liposomally encapsulated compound changes with respect to unmodified (control) liposomes. Often, the tumour to blood concentration ratio will be reduced with respect to the control, particularly at late time points (e.g. 24 to 144 h) and such reductions in tumour to blood ratios of formulations of liposones with enhanced tumour uptake due to enhanced circulation lifetime, are evident in several reports as discussed above.

However, simulations with mathematical modelling of biodistribution (TABLE A) show that tumour to blood ratios do not necessarily fall when uptake by an eliminating organ or organs is reduced. This is demonstrated by considering a 4 compartment model (compartment 1=blood; 2=tumour; 3=rest of tissues; 4=elimination organ(s)), and the way in which the tumour to blood concentration ratios change after a bolus intravenous injection of unmodified liposomes and test liposomes with a reduced uptake (K4, 1) by the elimination compartment.

TABLE A

| | |
|---|---|
| MODEL PARAMETERS USED FOR SIMULATION | TUMOUR-BLOOD RATIO |

| LD. | k2.1 | k1.2 | k3.1 | k1.3 | k4.1 | k1.4 | k0.2 | k0.3 | k0.4 | 3 h | 24 h | 48 h | 144 h |
|---|---|---|---|---|---|---|---|---|---|---|---|---|---|
| 049 | 0.015 | 0.01 | 0.8 | 0.06 | 1 | 0.06 | 0.0019 | 0.002 | 0.0019 | 0.3 | 0.7 | 1.1 | 1.8 |
| 048 | 0.015 | 0.01 | 0.8 | 0.06 | 3.9 | 0.06 | 0.0019 | 0.002 | 0.0019 | 0.2 | 0.7 | 1.1 | 1.9 |
| 047 | 0.015 | 0.01 | 1 | 0.06 | 1 | 0.06 | 0.02 | 0.002 | 0.011 | 0.3 | 0.6 | 0.8 | 0.9 |
| 046 | 0.015 | 0.01 | 1 | 0.06 | 3.9 | 0.06 | 0.02 | 0.002 | 0.011 | 0.2 | 0.6 | 0.8 | 1.1 |
| 045 | 0.015 | 0.01 | 1.2 | 0.06 | 1 | 0.06 | 0.02 | 0.002 | 0.011 | 0.3 | 0.6 | 0.7 | 0.9 |
| 044 | 0.015 | 0.01 | 1.2 | 0.06 | 3.9 | 0.06 | 0.02 | 0.002 | 0.011 | 0.2 | 0.6 | 0.8 | 1.1 |
| 043 | 0.015 | 0.01 | 0.1 | 0.06 | 1 | 0.06 | 0.02 | 0.002 | 0.0019 | 0.1 | 0.5 | 0.7 | 0.9 |
| 042 | 0.015 | 0.01 | 0.1 | 0.06 | 3.9 | 0.06 | 0.02 | 0.002 | 0.0019 | 0.1 | 0.5 | 0.7 | 0.9 |
| 041 | 0.015 | 0.01 | 0.1 | 0.06 | 1 | 0.06 | 0.0019 | 0.002 | 0.011 | 0.2 | 0.8 | 1.4 | 3.5 |
| 040 | 0.015 | 0.01 | 0.1 | 0.06 | 3.9 | 0.06 | 0.0019 | 0.002 | 0.011 | 0.1 | 0.9 | 1.8 | 5 |
| 039 | 0.015 | 0.01 | 0.8 | 0.06 | 1 | 0.06 | 0.02 | 0.002 | 0.011 | 0.3 | 0.6 | 0.8 | 1 |
| 39b | 0.015 | 0 | 0.8 | 0.06 | 1 | 0.06 | 0.02 | 0.002 | 0.011 | 0.3 | 0.7 | 1 | 1.5 |
| 39a | 0.015 | 0.01 | 0.8 | 0.06 | 1 | 0.06 | 0 | 0.002 | 0.011 | 0.3 | 0.85 | 1.3 | 2.8 |
| 038 | 0.015 | 0.01 | 0.8 | 0.06 | 3.9 | 0.06 | 0.02 | 0.002 | 0.011 | 0.2 | 0.6 | 0.9 | 1.1 |
| 037 | 0.015 | 0.01 | 0.1 | 0.06 | 1 | 0.06 | 0.02 | 0.002 | 0.011 | 0.2 | 0.6 | 0.9 | 1.2 |
| 37b | 0.015 | 0 | 0.1 | 0.06 | 1 | 0.06 | 0.02 | 0.002 | 0.011 | 0.2 | 0.7 | 1.2 | 2.2 |
| 37a | 0.015 | 0.01 | 0.1 | 0.06 | 1 | 0.06 | 0 | 0.002 | 0.011 | 0.2 | 0.8 | 1.5 | 4 |
| 036 | 0.015 | 0.01 | 0.1 | 0.06 | 3.9 | 0.06 | 0.02 | 0.002 | 0.011 | 0.1 | 0.7 | 1.1 | 1.4 |
| 031 | 0.015 | 0.01 | 0.8 | 0.064 | 1 | 0.06 | 0.02 | 0.25 | 0.011 | 0.3 | 1.5 | 2.4 | 5.9 |
| 31b | 0.015 | 0 | 0.8 | 0.064 | 1 | 0.06 | 0.02 | 0.25 | 0.011 | 0.4 | 1.8 | 3.5 | 18.6 |
| 31a | 0.015 | 0.01 | 0.8 | 0.064 | 1 | 0.06 | 0 | 0.25 | 0.011 | 0.4 | 2.1 | 5 | 39.4 |
| 030 | 0.015 | 0.01 | 0.8 | 0.064 | 3.9 | 0.06 | 0.02 | 0.25 | 0.011 | 0.3 | 1 | 1.5 | 2.2 |
| 029 | 0.01 | 0.01 | 0.8 | 0.064 | 1 | 0.06 | 0.008 | 0.25 | 0.011 | 0.1 | 1.1 | 2.6 | 14.2 |
| 028 | 0.01 | 0.01 | 0.8 | 0.064 | 3.9 | 0.06 | 0.008 | 0.25 | 0.011 | 0.1 | 0.8 | 1.6 | 4.2 |
| 027 | 0.01 | 0.01 | 0.8 | 0 | 2.5 | 0.06 | 0.008 | 0.25 | 0.011 | 0.3 | 1 | 2 | 6.7 |
| 026 | 0.01 | 0.005 | 0.8 | 0.064 | 1 | 0.06 | 0.008 | 0.25 | 0.011 | 0.2 | 1.4 | 3 | 24.7 |
| 025 | 0.01 | 0.005 | 0.3 | 0.064 | 3.9 | 0.06 | 1.008 | 0.25 | 0.011 | 0.2 | 0.9 | 1.8 | 6.5 |
| 022 | 0.01 | 0.01 | 0.8 | 0 | 1 | 0.06 | 0.008 | 0.25 | 0.011 | 0.4 | 1.4 | 3 | 20.2 |
| 021 | 0.01 | 0.01 | 0.8 | 0 | 3.9 | 0.06 | 0.008 | 0.25 | 0.011 | 0.2 | 0.9 | 1.6 | 4.6 |
| 005 | 0.01 | 0.003 | 0.8 | 0.064 | 1 | 0.1 | 0.006 | 0.001 | 0.02 | 0.2 | 0.5 | 0.9 | 2.2 |
| 05b | 0.01 | 0 | 0.8 | 0.064 | 1 | 0.1 | 0.006 | 0.001 | 0.02 | 0.2 | 0.6 | 1 | 2.8 |
| 05a | 0.01 | 0.003 | 0.8 | 0.064 | 1 | 0.1 | 0 | 0.001 | 0.02 | 0.2 | 0.6 | 1.1 | 3.6 |
| 02 | 0.01 | 0.01 | 0.8 | 0.064 | 1 | 0.06 | 0.008 | 0.25 | 0.011 | 0.2 | 1.2 | 2.5 | 13.3 |
| 01 | 0.01 | 0.01 | 0.8 | 0.064 | 3.9 | 0.06 | 0.008 | 0.25 | 0.011 | 0.2 | 0.8 | 1.5 | 3.9 |
| A four compartment model was constructed and simulations were performed using SCOMP (a Pascal program for IBM PC's written by M. S. Leaning and M. A. Boroujerdi (1991))[1]. The model has options for the kinetics of flux between compartments. Setting fluxes to have linear kinetics in all compartments except the elimination organ(s) and a Langmuir flux in the latter (to simulate a saturable clearance mechanism), gave realistic simulations of our own and other's data. | | | | | | | | | | | | | |
| #The rates shown in the table define the transfer rates between compartments. The hypothetical bolus input at time 0 was 100 in each case. Tumour:blood ratios were calculated using the model's output for the concentrations in the four compartments over time. | | | | | | | | | | | | | |

[1]Leaning M S, Boroujerdi M A: A system for compartmental modelling and simulation. Computer Methods & Programs in Biomedicine (1991); 35:71-92.

Depending on the settings of the parameters for the rate constants for transfer between compartments, this change (reduced K4,1) can produce an increment in tumour to blood ratio, a decrease, or a complex change (e.g. increase at early time points with decrease at late time points). Where the ratio of the elimination rates of the tumour and the rest of the tissues (KO2/KO,3) is high (e.g. 10) the tumour to blood ratio tends to fall and when it is low (<<1) the tumour to blood ratio conversely tends to rise, with a complex change being observed at intermediate values. In addition, where the ratio of the elimination rates for the rest of the tissues and the elimination organ(s) (KO,3/KO,4) is low or high, tumour to blood ratios tend to fall and rise respectively with reduced K4,1. Note, however, that where this ratio is >1, compartment 4 is not the predominating elimination organ, thus this scenario is not relevant to liposomal modifications which exclude liposomes from the RES. The other factor influencing whether tumour to blood ratios rise or fall with reduced K4,1 is the ratio of the entry rates for the rest of the tissues and the elimination organ(s) (K3,1/K4,1), the outcome depending on the ratios before and after modulation of K4,1. This ratio has a much less predictable effect on tumour to blood ratio (i.e. various changes which alter this ratio in the same direction can have different effects on the direction of change of the tumour to blood ratio), but using parameters in multicompartment models that fit the behaviour of control liposomes, reduction of K4,1 without concomitant alteration of other parameters tends to lead to reduced tumour to blood ratios. Thus, with the exception of situations where the tumour has a much lower destruction rate than other “non-elimination” organs (KO2<<KO3), the tumour to blood ratio tends to fall as the rate of uptake of the liposomes by the elimination organ(s) decreases. It will be recalled that this is an undesirable effect of modifications of the liposomes to exploit the push mechanism.

In contrast to the above scenarios, any modification reducing the destruction rate of liposomes by the tumour tissue, or reducing the egress rate from the tumour back to the blood, will increase tumour to blood ratios. Thus the three scenarios where tumour to blood ratios always rise (at the same time as the concentration of liposomes in the tumour increases) following liposome modification are:

1) where the destruction rate of liposomes by the tumour is reduced by the modification.

2) where the transit rate of liposomes from the tumour back to the blood is reduced by the modification;

3) where the uptake rate into the elimination organ(s) is reduced in the modified liposomes and where, with both the modified and modified liposomes, the tumour has a much lower destruction rate than other “non-elimination” organs (KO,2 <<KO,3).

Each of these scenarios provides an increment in tumour to blood ratio which alone or together can reduce or eliminate the tendency for tumour to blood ratios to fall when reduced entry into elimination organ(s) occurs simultaneously with the above changes. The failure of the tumour to blood ratio to fall, when there is reduced entry into elimination organ(s), occurs when there is reduced destruction within or egress from the tumour and is an important discriminant between the “push” and “trap” principles.

Previous pharmacokinetic studies have shown that conventional liposomes exhibit similar dose-dependency for both the degradation rate constant and uptake rate constant for liposomes and suggested therefore that there was the same underlying mechanism for both uptake and degradation [Harashima et al Biopharamaceutics and Drug Disposition, 14: 265-270, (1993)]. Thus the prior art discourages the notion (which is the foundation of the present invention) that PEG at different doses and/or different formulations of liposome can have an independent impact on both the uptake and degradation processes; the two processes in fact cannot have the same underlying mechanism since they are now seen to be modulated independently.

In contrast to the prior art, the principles of the present invention lead to a different method for optimisation of liposomes and also give ways of discriminating between liposome modifications merely operating by a push principle and those which enhance tumour to blood ratio via a trapping mechanism. Thus optimisation in accordance with the invention abandons the improved circulation time and the exclusion from the reticuloendothelial system (liver and spleen) as the arbiters of modified liposome function.

In order to produce liposomes (or other lipid-based structures) in which tumour (or skin) delivery is enhanced by exploiting the trap mechanism, it is necessary to consider and optimise a variety of interdependent aspects of the liposome (or other lipid-based structure) material. In outline, for liposomes, decisions are required in relation to at least the following features:

1. The size of the liposomes.

2. Whether to use uni- or multi-lamellar liposomes.

3. The composition of the lipid components, both in terms of the individual species of lipids to be used and the relative proportions thereof.

4. The degree and nature of PEG-modification.

These features will be discussed in greater depth below. Suffice to say at this point that the optimisation is to a certain extent a question of trial-and-error for each target tissue (tumour-type, or skin), diagnostic or therapeutic agent and PEG-modified liposome (or other hydrophilic moiety-modified lipid-based structure) formulation. However, elucidation of the principles behind the invention enables a series of tests to be identified, and criteria established which will both enable the necessary optimisation to be conducted and the discrimination of liposomes or other lipid-based structures which exploit the trap mechanism from those which merely operate on the push principle.

In order to distinguish between lipid-based structures which exploit the trap mechanisms of the present invention and those which do not, it is necessary to make comparisons between the performance of the lipid-based structures in question (the “test species”) and two control products which are identical in all respects to the test species save as follows:

(a) first control product: this differs from the test species only in that it lacks any hydrophilic moiety modification;

(b) second control product: this differs from the test species in that it lacks any hydrophilic moiety modification and in that it lacks any lipid components which have the capacity to be modified by attachment of hydrophilic moieties in the test species.

These requirements may best be illustrated by an example. Say the test species is a liposome which comprises a therapeutic agent entrapped in unilamellar vesicles of a given size formed of a mixture of two lipid species (A and B) which cannot be PEGylated and a lipid species (C) which has the capacity to be PEGylated and, in the test liposomes, is PEGylated. The first control product will thus also be a unilamellar liposone, of the same size and content of therapeutic agent and formed of the same mixture of lipid species A, B and C as the test liposome (but species C is not PEGylated). The second control product will thus also be a unilamellar liposome of the same size and content of therapeutic agent and will be formed of a mixture of lipid species A and B only in the same relative proportions as for A and B in the test liposomes.

In another example, the liposome is composed of a single lipid specie which is susceptible to PEGylation or of two or more lipid species, each of which is susceptible to PEGylation. Only a portion of the lipids in the test liposomes is PEGylated. In this case the first control product is formed of the same lipid specie or combination of lipid species as the test liposomes but now there is no PEGylation. The second control product is, in these special types of case, replaced in the comparison tests by the first control product.

The test is conducted by intravenous injection of a standard dose of a diagnostic or therapeutic agent (hereafter the “agent”) entrapped in the various liposome products into test animals with an appropriate model solid tumour. The blood and tumour concentrations of the agent are measured at 24 and 48 hours after the injection.

For liposomes according to the present invention the ratio of tumour concentration of the agent to the blood concentration of the agent achieved at either or both the 24 and 48 hour points will be greater than unity.

Moreover the ratio of tumour concentration to blood concentration achieved by liposomes of the present invention will not be significantly lower at either 24 or 48 hours than the tumour to blood concentration ratio achieved by the first control product.

In addition the tumour concentration at each of the 24 and 48 hour points achieved by liposomes of the invention will be greater than the tumour concentration at the same time points achieved by the first control product and also, where it is appropriate to compare with a second control product, greater than the tumour concentration at the same time points achieved by the second control product.

As previously mentioned, optimisation of delivery of diagnostically and therapeutically effective agents to tumours or the skin by other lipid-based structures bearing hydrophilic moieties may be achieved by application of these principles in the same way as described above in relation to the use of PEGylated liposomes for delivery of agents to tumours.

The present invention therefore provides a composition of a diagnostically or therapeutically effective agent for administration via the bloodstream to a solid tumour or the skin, the composition comprising a lipid-containing multi-molecular structure, the agent being present predominantly in the lipid-containing multi-molecular structure, wherein the lipid-containing multi-molecular structure comprises one or more hydrophobic entities bearing covalently bound hydrophilic polymer moieties, and wherein the physical form of the lipid-containing multi-molecular structure, the nature of the hydrophobic entities, the nature of the hydrophilic polymer moieties, the ratio of the polymer-bearing hydrophobic entities to non-derivatised hydrophobic entities exposed to the bloodstream and, when there are two or more hydrophobic entities, the relative proportions of the hydrophobic entities, are all selected such that:

(i) on intravenous injection of the composition to an animal, where appropriate bearing a model solid tumour, the ratio of tumour concentration to blood concentration or the ratio of skin concentration to blood concentration of the agent achieved at either or both of 24 and 48 hours following the injection is greater than unity,

(ii) the ratio of tumour to blood concentrations or the ratio of skin concentration to blood concentration of the agent achieved at 24 and 48 hours following intravenous injection of the composition to an animal, where appropriate bearing a model solid tumour, is not significantly lower than the ratio of tumour concentration to blood concentration or that the ratio of skin concentration to blood concentration of the agent achieved at the same times after intravenous injection to an animal, where appropriate bearing a model solid tumour, of a first control product, which is identical to the composition except that the first control product lacks any hydrophilic polymer modification of the hydrophobic entities,

and

(iii) except in the case where the composition consists essentially of an agent associated with a lipid-containing multi-molecular structure consisting of one or more species of hydrophobic entity, each specie being susceptible to derivatisation with hydrophilic polymer moieties and where at least a portion of each of the species of hydrophobic entities is derivatised with hydrophilic moieties, the tumour concentration or skin concentration of the agent achieved by intravenous injection of the composition to an animal, where appropriate bearing a model solid tumour, is greater at 24 and 48 hours following injection than is the tumour concentration or skin concentration of the agent achieved by intravenous injection to an animal of a second control product, which is identical to the composition except that the second control product lacks any hydrophilic polymer modification and lacks any hydrophobic entities which are derivatised by polymer modification in the composition, or, in the case where the composition consists essentially of an agent associated with a lipid-containing multi-molecular structure which consists of one or more species of hydrophobic entity, each specie being susceptible to derivatisation with hydrophilic polymer moieties and where at least a portion of each of the species of hydrophobic entities is derivatised with hydrophilic moieties, the tumour concentration or skin concentration of the agent achieved by intravenous injection of the composition to an animal, where appropriate bearing a model solid tumour, is greater at 24 and 48 hours following injection than is the tumour concentration or skin concentration of the agent achieved by intravenous injection to an animal of the first control product as defined above.

There are no limits imposed in general on the therapeutically and diagnostically effective agents which may be delivered by the compositions of the invention except in the sense that the agent will, of course, be one intended to be effective either in treating or diagnosing solid tumours, where the composition is optimised for delivery of the agent to a tumour, or else for treating or diagnosing skin diseases or disorders of the skin when the compositions have been optimised for delivery to the skin. By way of example, agents which may be administered in compositions of the present invention include drugs, for instance cytotoxic and cytostatic drugs, and nucleic acids, especially DNA.

The amount of agent in the compositions of the invention will be selected to be effective in the intended therapy or diagnosis. The compositions will generally provide the same dose at the target site as a conventional treatment or diagnostic composition of that agent, or possibly less than the conventional dose when the “trapping” achieved by the composition enhances the efficacy of that agent. Doses greater than the conventional dose may be delivered when this is clinically desirable and where conventional doses are limited by toxic effects not experienced with the compositions of the invention or by the inability of conventional administration forms to deliver desired doses of the agent to the target tissue.

As used herein the term “multi-molecular structure” is intended to encompass any structure comprising an assemblage of similar molecules or of dissimilar molecules which is stabilised by covalent or non-covalent bonding, for instance hydrogen bonding or hydrophobic interactions. The multi-molecular structures must contain at least one lipid specie and this requirement is reflected in the use of the term “lipid-containing multi-molecular structure”. It should be noted that the mine requirement for one lipid specie to be present in these structures may be satisfied by the presence of a lipid specie as the “hydrophobic entity” bearing hydrophilic polymer moieties also required as a part of the structures of the invention. The nature of the multi-molecular structures used in the compositions of the invention, such as liposomes will be discussed below, as will the nature of the hydrophobic entities, such as lipids and particularly phospholipids and the nature of the hydrophilic moieties, such as polyethylene glycol residues. For brevity the lipid-containing multi-molecular structures of the invention are herein generally referred to as “lipid-based structures” and the two terms should therefore be regarded as inter-changeable.

As regards the physical form of the multi-molecular structures, there are no particular limits imposed by the present invention. The physical form adopted will, however, often be dictated by the nature of the target tissue for treatment or by the nature of the hydrophobic entities selected for use in the compositions. In some cases the physical form of the structures will be dictated by intereactions between the therapeutic or diagnostic agent and the hydrophobic entities. Thus, for instance, with certain drugs, lipids tend to form drug-lipid complexes in the form or ribbons or discoids. The agent may therefore be present, for instance, entrapped within the lipid-based structures or otherwise bound to the lipid-based structures.

The relative proportions of hydrophobic entities which are not derivatised and of those which are derivatised with hydrophilic polymer moieties affects the surface properties of the multi-molecular structures as seen by the patient's tissues. Accordingly it is the ratio of these two types of components exposed to the bloodstream that affects the performance of the compositions of the invention. There is no particular requirement imposed by the present invention on the proportions of these different types of component in parts of the multi-molecular structures not exposed to the bloodstream. Thus, for instance, in the case of liposomes of the invention it is permissable to have assymetry between the composition of the blood-contacting external surface lipid layer of the liposomes and the composition of the internal surface lipid layer of the liposomes.

Where there are two or more species of hydrophobic entities in the compositions of the present invention, the relative proportions of the various species of hydrophobic entities can be adjusted and optimised to provide the desired “trapping” of the agent in tumours or skin. There are no specific limits placed on the number of different species of hydrophobic entities, nor on the proportions of each species in the composition.

As regards model tumours, since experiments for optimisation of compositions cannot normally be conducted on humans, it will be necessary to select for use in the optimisation of compositions of tumour therapeutic or diagnostic agents in accordance with the principles of the present invention, experimental animals which bear solid tumours which are representative of the human tumours which are to be treated by the optimised compositions of the invention. Similarly, for optimisation of compositions for treatment or diagnosis of dermatological diseases and defects, it will be necessary to select experimental animals which have skin which is a good model of human skin. All experimental animals should also be good models of humans as regards the pathways used for delivery of the compositions to the target tissues and as regards elimination and destruction of the therapeutic or diagnostic agents. This will often constrain the choice of experimental animals in which to conduct the necessary tests.

The animals referred to above are suitable species and strains of animal, preferably conventional laboratory animals such as rodents or primates, selected as models for the human therapy or diagnosis for which the agent and the composition of the invention are intended. Naturally, the animals used for administration of the composition of the invention and for the administration of the first control product and, where appropriate, the second control product will be substantially identical and will certainly be matched in accordance with normal laboratory practice. Usually animal tests will be conducted on groups of animals of appropriate numbers to secure statistically meaningful data from the experiments.

As regards item (ii) above, it is of course possible that the measured concentration ratio achieved with compositions of the invention will be greater at either or both of 24 and 48 hours than that achieved with the first product: this is preferred. However it is also possible that compositions according to the invention will give at one or both of the 24 and 48 hour time points a measured concentration ratio which is numerically less than that achieved using the first control product. This is also acceptable within the present invention (although it is less preferred) provided that the difference between the measured concentration ratios achieved with the composition of the invention and the first control product is not statistically significant. Appropriate statistical tests of significance are readily available to those skilled in the art but should be selected having regard to the experimental protocol for measuring the concentrations of the agent.

It is especially preferred that the tumour or skin concentration of the diagnostically or therapeutically effective agent achieved by administration of the composition of the invention remains greater than the blood concentration achieved by administration of the composition throughout the period from 24 to 48 hours after administration.

The exception in item (iii) above ensures that there is an appropriate comparison available for all possible embodiments of the present invention since a “second control product” as defined above lacking all derivatisable hydrophobic entities would necessarily lack any lipid component in the case dealt with by the exception.

In accordance with the present invention compositions for delivery of a particular agent to a particular type of solid tumour or to skin may be optimised by iterative steps of testing the candidate composition as described above, then modifying one or more features of the composition, retesting and further modification directed by the results of the retesting. The objective of optimisation will depend to some extent on the intended use of the agent in question. In the case of a therapeutic agent the most important parameter is the tumour or skin concentration of the therapeutic agent since maximising this will result in more effective delivery to the tumour or skin. The duration for which the agent remains in the tumour or skin is also a factor to be considered in maximising the overall dose delivered from a single administration of the agent. In the case of a diagnostic agent the overall dose administered may be less significant, and the duration for which the agent remains in the tumour or skin may also be almost irrelevant. What is most important usually is to ensure a high contrast between the tumour and the non-tumour tissues surrounding the tumour tissues or between the skin and other tissues and this will usually be achieved by optimising for high tumour to blood concentration ratios or skin to blood concentration ratios.

In optimising the compositions either for tumours or for skin, therefore, one will generally examine two properties:

1) the tumour to blood ratio or skin to blood ratio as appropriate; and

2) the tumour concentration or the % injected dose per gram of tumour, at an appropriate selection of time points (e.g. 3 h, 24 h, 48 h, 72 h, 144 h) , or equivalent measurements for skin as appropriate.

Alternatively, where appropriate animal models are available, tumour or skin destruction rates could be measured directly.

In the discussion to follow, reference is made to liposomes as examples of lipid-based structures that may be used in the present invention and to PEG moieties as examples of the hydrophilic moieties which may be used in accordance with the present invention. The discussion below refers especially to the delivery of diagnostic and therapeutic agents to tumours but is also applicable to delivery of diagnostic and therapeutic agents to skin, for instance in order to detect or treat dermatological disorders.

In the absence of direct measurements, evidence that a modification is enhancing “trapping” (and is not just improving circulation time via reduction of entry rate into an elimination organ) comes from two sources:

1) the lack of a decline in tumour to blood ratios or skin to blood ratios relative to the first control product; and

2) the observation that the extent of liposome modification maximising blood levels is often different from that maximising the % dose in the tumour at any given time point and/or maximising the tumour to blood ratio or equivalent observations for the skin.

Where the underlying formulation (with or without PEG-modification) enhances trapping, reduced entry into an elimination organ via the PEG modification will increase tumour to blood ratios. Thus, optimisation of the unmodified liposome to maximise trapping is via examination of the impact of modifications that reduce entry into elimination organs (since, with good trapping, this results in tumour to blood ratios increasing with modification whereas, with poor trapping these ratios decline). The same rationale applies to the skin.

### Lipid-containing Multi-molecular Structures

The compositions of the present invention comprise a lipid-based structure, which may be in the form of liposomes or other lipid-based, especially phospholipid-based, structures such as micelles and other structures mentioned below.

DNA exposed to cationic liposomes has been demonstrated to form “spaghetti-like structures” apparently extruding from the liposomes with time (filamentous lipoidal DNA). This structure is thought to be due to fusion of liposomal bilayers round DNA such that a strand of duplex DNA becomes coated with a lipid bilayer. Other forms of DNA/lipid complexes have been observed, and are thought to be similar to hexagonal II phase lipids, but with DNA present in the 5 nm lumen of the hexagonal tubes of lipid. These tubes are packed together with their hydrophobic surfaces in contact and each bundle of tubes has an exterior lipid coat orientated with its hydrophilic surface facing the aqueous environment (such structures may lie within portions of the lipid bilayer of a liposome).

Non-liposomal drug complexes have also been described. The antifungal agent Amphotericin-B has been shown to form ribbon-like structures (ABLC™, The Liposome Company) with DMPC and DMPG (7:3) and discoidal structures with cholesterol sulphate. Thus, not all drug/lipid complexes are liposomal. However, depending an size and the nature of the external lipid surface, such non-liposomal lipid carriers will share many of the properties of liposomes. The methods used to attach polymers to liposomes can readily be applied to attach polymer to appropriate lipid complexes.

### Hydroahobic Entities

The hydrophobic entities envisaged for use in the present invention are generally lipids but there other types of hydrophobic entities which may be used, for instance hydrophobic peptides, polypeptides and proteins. The main requirement is that the entities are sufficiently hydrophobic to be retained in the lipid-based structure of the compositions of the invention whilst the therapeutic or diagnostic agent is delivered to and entrapped within the target tissue. Some of the hydrophobic molecules in the compositions of the invention are also required to act as anchors for the hydrophilic polymer moieties which are presented on the blood-contacting surfaces of the compositions; these are discussed below in connection with the hydrophilic polymer moieties.

### Hydrophilic Polymer Moieties

The polymer moieties may be bound to any hydrophobic molecule which can be integrated into the lipid-based structure so as to anchor the polymer in the lipid-based structure, provided that the lipid-based structure is not thereby disrupted, that the anchor molecule bears a suitable reactive group able covalently to bind the polymer and that the anchor molecule is not readily lost from the lipid-based structure. Preferably the molecule used to anchor the polymer moiety is a phospholipid. The polymer may be bound to the anchoring molecule by any known covalent bonding technique, preferably involving binding the polymer to an amino group of the anchor molecule. The bond should, in addition to being covalent, be non-biodegradable in normal blood or serum for the intended duration of residence in the bloodstream (usually at least 24 h and preferably 48 h), non-toxic and non-immunogenic. Where the hydrophilic moieties are bonded to phospholipids as anchor molecules, the phospholipids and the bond to the hydrophilic moieties should preferably be phospholipase resistant. The use of TMPEG (see WO-A-90/04384, WO-90/04606, WO-A-90/04650 and WO-A-95/06058) is preferred for coupling PEG moieties to the phospholipid. These techniques may be applied before or after assembly of the liposomes or other lipid-based structure according to the suitability of the individual method chosen and the desired product. Where liposomes bearing polymer moieties are to be used, the polymer moieties may be added before liposome formation but this tends to reduce the internal space available for carrying the payload and increases the amount of polymer required to achieve the desired coverage of the external surface. It is, of course, the amount of polymer exposed to the blood stream or tissues at the external surface of the liposomes or other lipid-based structure forming the dispersed phase, which primarily influences the tumour localisation of the payload. The present invention places no particular limit on the quantity of hydrophilic polymer moieties to be exposed on the blood-contacting surface of the lipid-based structures other than the requirements imposed by the optimisation of the composition for its intended use. However, in general, it is preferred that at least 2% of the hydrophobic molecules in the blood-contacting surface of the lipid-based structures are derivatised with hydrophilic moieties, especially in the case of liposomes. Provided that they are all derivatisable, up to 100% of the hydrophobic entities exposed at the blood-contacting surface of the lipid-based structures may be so derivatised if desired or necessary to achieve the therapuetic or diagnostic goal. Where only some of the hydrophobic entities exposed at the blood-contacting surface are derivatised it is preferred that the content of the derivatisable hydrophobic entities and the degree of derivatisation are such that at least 2% of the total hydrophobic entities exposed at the blood-contacting surface are derivatised with hydrophilic moieties. More preferably from 20 to 100% of the derivatisable hydrophobic entities are actually derivatised. [The degree of derivatisation of hydrophobic entities by hydrophilic moieties is herein expressed as mole % except where the context requires otherwise.] The amount of hydrophilic moieties on hydrophobic entities not exposed to the blood-contacting surfaces of the lipid-based structures is less critical. It may be convenient that the proportion of the hydrophobic entities which are derivatised with hydrophilic polymer moieties will be substantially constant throughout the lipid-based structures of the invention and in the case where all the derivatisation of the hydrophobic entities is conducted before assembly of the lipid-based structures, the production process makes it almost inevitable that the composition of the lipid-based structures will be substantially constant throughout the lipid-based structures.

The polymer moieties may be of any suitable hydrophilic polymer though preferably polyethylene glycol (PEG) is used, especially PEG's of molecular weight from 250 to 12000 and more preferably PEG 5000.

In the compositions of the invention the diagnostic or therapeutic agent is at least partially associated with the lipid-based structures. Preferably at least 50% by weight of the diagnostic or therapeutic agent is associated with the lipid-based structures, for instance by entrapment within or between the lipid bilayers or in the enclosed aqueous environment of liposomes or incorporated into the lipid bilayer thereof.

The invention will be described further below with reference to liposomal embodiments and to PEG as the polymer but the principles outlined above and utilised below may equally be applied to other phospholipid-based compositions of the invention.

The experiments given below demonstrate that the optimisation for retention in the blood and exclusion from elimination organs (disclosed in the prior art) does not yield a fully optimised liposome with respect to tumour retention and the prior art shows that the polymer may actually worsen tumour to blood ratios.

The experiments illustrate optimisation, based on either an empirical study of tumour to blood ratios in conjunction with tumour liposome concentrations, or direct measurements of flux rates from tumours and elimination rates within tumours, and thus allow the construction of a liposomally entrapped compound with optimum retention within tumours and acceptable tumour to blood ratios.

Since tumours vary qualitatively and the blood/tumour barrier is not always identical, it is not possible to disclose a single formulation with optimunm properties. This will have to be determined for different types of tumour individually. However, given the principles discussed above and outlined below and the demonstration of the applications of those principles in the Examples, optimum tumour retention times and tumour to blood ratios can be achieved for any particular tumour.

i) PEG dosage

In Example 9 (FIGS. 2 and 4), there is up to 21 & 40 mol % of PEG-PE on the exterior surface (depending on the proportion of the PE that became PEGylated). In Example 4, the range 5% -100% is explored. An earlier report [Tilcock et al, Biochem. Biophys Acta 110:193-198, (1992)] suggested that of 20 mol % phosphatidyl ethanolamine only 7 mol % of the total surface lipid became PEGylated (under specified conditions). However with longer reaction times, incremental addition of TMPEG to overcome hydrolysis, and/or greater molar excess of TMPEG there is no a priori reason that all the PE could not be PEGylated since Blume & Cevc (Biochim. Biophys. Acta, 1146:157-168, (1993)) could produce 100% PEG-PE liposomes by incorporation. This has demonstrated that 100% packing is feasible, but one cannot exclude a kinetic problem in the presentation of the PEG chains to the surface.

All previously reported uses of PEG-liposomes for tumour drug delivery make their liposomes by incorporation of PEG-lipid. Allen et al (Biochim. Biophys. Acta, 1066:29-36, (1991)) showed that less PEG-PE was incorporated into the liposomes than was added to the lipid composition. With PEG-1900-PE at 10% only 5.7, 5.0, 6.8, 6.5 mol % became incorporated into liposomes and the comparable figures for PEG-5000 were 6.9 and 7.3 mol %. Since it was noted that foaming of the lipid mixture occurred with higher mol % of PEG(1900)-DSPE and that foaming could be removed by cbromatography, the failure to incorporate all the PEG-PE was attributed by Allen et al (1991) to formation of PEG-DSPE micelles. Foaming actually provides a potentially important environment where the air/water interface may, by providing an alternative to the bilayer environment, discourage incorporation of the foam-entrapped free PEG-lipid into the bilayer. Whether this putative foam-entrapped PEG-lipid or the putative micelles of PEG-PE are more important remains to be established.

It should be noted that the results presented in the Examples given below contrast with those of Allen et al (1991), where a similar limit of PEGylation with PEG-750, PEG-1900 and PEG-5000 and maximum blood retention with PEG-1900 was observed. The inventors were able to examine a much wider range of PEG substitutions and found significant reduction in hepatic uptake after increasing the target PE for PEGylation from 5 to 40% with concomitant changes in blood levels.

ii) linkage of PEG to PE:

1) The preferred linkage is more stable to enzymatic degradations than ester or amide linkages which are subject to cleavage by esterases and anidases respectively. Succinyl ester linkages may additionally undergo hydrolysis (Carter and Meyerhoff, J. Immunol. Methods, 1985, 81, 245-257). With biodegradable linkages, degradation in the tumour milieu might ensue. A variety of linkages have been assessed (as micelles) for stability in serum [Parr et al, Biochim. Biophys. Acta, 1195: 21-30,(1994)]. Succinate-linked PEG-lipid, which contains an ester bond, was most susceptible to loss of PEG. Carbamate-linked PEG-lipid was much less unstable but a very small amount was still lost over 24 hours. Amide linkages appeared stable under these conditions (which presumably lacked amidases) and PEG-lipid in which the polymer was linked directly to the phosphate head group of phoshatidic acid showed intermediate stability between ester linked and carbamate-linked lipid. All four PEG-lipids were also subject to degradation via loss of one or both of the acyl chains (Parr et al, 1994). In addition to these considerations, it should be appreciated that the stability of the anchorage of the PEG-lipid in the bilayer is also relevant and related to the nature of the fatty acyl chain. Given the results reported by Parr et al (1994), it is anticipated that the PEG-lipid would be lost more rapidly from the DOPE:DOPC liposomes than the DSPE:DSPC liposomes of the Examples.

2) The preferred linkage should not generate a net negative charge when the amino group is substituted, in contrast to other linkages, such as the carbamate linkage which has been shown by Woodle et al, (Biophysical Journal, 61:902-910, (1992)) to consume the positive charge of the NE₂ group, leaving phosphatidyl ethanolamine with a net negative charge.

Significantly, groups using carbamate linked PEG-lipids have reported substantial micelle formation (Allen et al, (1991)) which according to these workers limits the amount of PEG-PE that can be incorporated into the liposome. Beddu-Addo et al (Liposome Research Days Conference, Abstract A-19 (1994)) have reported phase separation and micelle formation, again with a limitation of the amount of PEG-lipid that can be incorporated into intact liposomes. In contrast, using PEG-lipid generated by the cyanuric chloride method it was found that the amount of PEG-lipid that could be incorporated was not limited, nor was micelle formation a problem (Blume and Cevc, (1993)). Although Blume and Cevc (1993) attributed the difference between their results and those of Allen (1991) to the effects of either cholesterol or lipid concentration, the results presented below show insufficient foaming (which Allen found was due to micelle formation) to prevent incorporation of high ratios of PEGylated DSPE or DOPE. What the linkage obtained by use of TMPEG and the triazine ring linkage resulting from the cyanuric chloride method have in common is that both conserve the positive charge of the PE head group and thus do not generate a net negative charge although there is some confusion on this point in the literature: Blume and Cevc, (Biocbim. Biophy. Acta, 1029:91-97, (1990)); Blume and Cevc (1993). The carbamate method, by generating a net negative charge of the PE head group, will alter the effective size and reduce the ability to close pack the head groups (via electrostatic repulsion). Since micelle formation is favoured by lipids that are relatively cone shaped (with the head groups being the base of the cone) the relative enlargement of a head group of an essentially cylindrical lipid (which would tend to form bilayers) would be anticipated to increase micelle formation. This notion of the effect of the net negative charge favouring a higher curvature is suggested by the observation that cholesterol allowed more negatively charged PEG-PE to be incorporated (Beddu-Addo et al, (1994)).

In addition to these considerations, the generation of a net negative charge on the lipid by PEGylation may also be disadvantageous because it might render the liposome susceptible to removal by macrophages (which have a scavenger receptor which takes up liposomes having a negative charge [Nishikawa et al. J. Biol Chem., 265: 5226-5231, (1990)]. Thus some of the benefit of PEGylation (which impedes macrophage uptake) may be offset by a PEGylation method that generated a net negative charge on the lipid.

Factors influencing tumour-retention of liposomes differ from those slowing blood clearance rates:

1) A slowing of blood clearance rate can be achieved by altering the lipid composition of the liposome but this does not in itself necessarily produce significant entrapment in tumours (even though it will, if the liposome size is appropriate, deliver more of the liposomal contents to the tumour than will more rapidly cleared liposomes of equivalent size).

2) When polymers such as polyethylene glycol are linked to the liposome exterior surface (or both surfaces), there is modest to marked increase in circulation time. However, although the impact of PEGylation may be proportionally greater for short-lived liposomes, the half life achieved will probably not exceed that of PEGylated long-lived liposomes (i.e. liposomes in which both lipid composition is optimised and polymer is added). Thus, to achieve a very long lived liposome, both an appropriate lipid composition and PEGylation have tended to be used. Since with a typical lipid composition giving long-lived liposomes, the attachment of PEG to the surface gives little improvement in half-life (and this requires relatively little PEG substitution), relatively modest degrees of PEGylation (1-20 mol % of PEG lipid on the exterior surface, or both surfaces) have been selected as those achieving maximum half-life. However the blood clearance rate and tumour clearance rate involve different factors, thus the consequences of a particular lipid composition and degrees of polymer substitution may have different impacts on the two rates.

3) Blood clearance of liposmally entrapped agents depends on multiple factors which have been reviewed extensively [Senior, Crit. Rev. Ther. Drug Carrier Syst. 3(2): 123-93 (1987)].

i) Adsorption of EDL, subsequent lipid exchange and consequent leakage. VLDL or LDL do not appear to have the same function. Fluid liposomes undergo this exchange more than relatively rigid liposomes. Cholesterol in liposomes which are relatively fluid at 37° C. and which show reduced fluidity with cholesterol also show reduced HDL induced leakage. This also occurs in other formulations with added lipids incorporated to reduce fluidity. In addition to direct transfer between liposomes and HDL, several other factors may participate in this disruptive lipid transfer to HDL:

(1) phospholipid transfer factors;

(2) apoproteins;

(3) lecithin cholesterol acyl transferase.

(ii) Adsorption of other serum proteins. This probably includes antibodies, complement and clotting factors (which bind negatively charged liposomes, although the latter may have no significant effect on clotting factor levels in vivo. Antibody or complement coating of autologous red cells makes them spleen and liver seeking respectively.

(iii) Reticuloendothelial system uptake is one of the major routes of elimination and is a saturable process (as indicated by the capacity for RES blockade).

(iv) leucocyte phagocytosis (macrophage, monocyte) and receptor mediated endocytosis (lymphocytes)

(v) zytic attack by lipases is also feasible.

(vi) The rate of tissue distribution will also influence blood clearance times (as with any other pharmaceutical).

(vii) Lipid exchange not via HDL (e.g. plasma/bilayer exchange, cell membrane/bilayer exchange).

(viii) Endocytosis into non-phagocytic cells has also been observed. PS:Chol 2:1 liposomes were shown to enter via endosomes into a low pH compartment [Straubinger et al, Cell, 32: 1069, (1983)].

Point (vi) is of particular relevance to the previously recommended optimisation procedure disclosed for “tumour-localising liposomes” (PCT/US 90/06211)). This factor potentially has a large impact on half life, but factors that delay entry into the tissues and hence reduce the tissue distribution rate, might well also reduce the rate of entry into the tumour, with consequent reduction of tumour to blood ratios.

(4) In contrast, many of the factors operating on plasma clearance will have little impact on the tumour destruction and/or egress rate. In addition, cancer cell membranes have been reported to have some differences in lipid compositions and rigidity compared with normal counterpart cells. Blitterswijk (in Physiology of Membrane Fluidity, ed. M Shinitzky (vol II) page 53-83,(1984), CRC Press Inc. Boca Raton, Fla.) reviewing this, provides an explanation for why both increases and decreases in membrane lipid fluidity have been reported. However there do appear to be differences in the cholesterol per surface area and in cholesterol:phospholipid ratios. As a consequence, lipid exchange in the tumour environment may not have the same consequences as in the blood. Thus, if longevity in the blood were to be partially achieved by lipid composition, that might be substantially altered (e.g. by cholesterol loss) in the tumour milieu.

5) The impact of PEGylation is also likely to be different if the major influences on clearance rates for the two sites (blood and tumour) have different mechanisms. Uptake by large organs, such as the skin, has the major impact on tissue distribution rate, hence on blood levels at early time points, whereas uptake by elimination organs such as liver and spleen influences the overall elimination rate and hence blood levels. Previous experience indicates that with long lived liposomes it has been reported that 3.5-7.5 mol % PEG-lipid produced optimum reduction in hepatic uptake and retention in the blood [Klibanov et al, Biochim. Biophys. Acta., 1062:142-148, 11991)]. Using liposomes with up to 80% substitution with PEG-lipid, Blume and Cevc (1993), showed that half life was maximal at 15 mol % in DSPC liposomes and 20% for sphingomyelin-containing liposomes, and was lower at both higher and lower degrees of substitution. Both observations suggest that this degree of PEG substitution is adequate to reduce extensive hepatic uptake, but the information pertaining to the differences in half life relating to the underlying lipid composition suggests that lipid composition still has an impact on either hepatic uptake or one of the other factors influencing blood clearance [Klibanov et al., (1991)]. The rather sparse PEGylation recommended by this optimisation rationale may be insufficient for optimisation of entrapment within tumours. For example it may be important to prevent almost all hydrophobic interactions with the surface. However, Cevc suggests that with high levels of polymer substitution the ends of the polymer reconstruct a hydrophilic surface to which proteins will attach (as opposed to a relatively mobile covering of less densely packed polymer chains) and hence the relationship between blood clearance and polymer degree of substitution at the surface is not, in his experience at least, monotonic (i.e. improves then declines with increasing polymer substitution) due to first diminution of hydrophobic interactions then increasing hydrophilic interactions. Thus the prior art not only teaches that the maximum benefit is achieved with comparatively little PEG substitution, but actually claims that further addition of PEG will be deleterious.

Size range of liposomes:

The three main types of capillary offer qualitatively different barriers to liposomes [Hwang in Liposomes: from Biophysics to Therapeutics, page 109, (1987), ed. M. J. Ostro, Marcel Dekker].

Continuous capillaries offer three routes across the endothelium:

i) pinocytic vesicle shuttle (50 nm particles);

ii) intercellular junctions (2-6 nm width);

iii) transendothelial channels 50 nm diameter and a basal lamina with pores of 5-10 nm in size.

Fenestrated capillaries offer four routes across the endothelium:

i) pinocytic vesicle shuttle (5-30 nm particles);

ii) diaphragm fenestrae (porosity unknown);

iii) Open fenestrae (40-60 nm) and

iv) intercellular junctions (4 nm).

Discontinuous capillaries offer two routes:

i) pinocytic vesicles (50 nm) and

ii) interstitial spaces (100-1000 nm); there are no basal lamina.

In experiments with 60 nm SUV and 400 nm MLV, liposomes did not cross the continuous capillaries of lung or skeletal muscle. This analysis suggests that the leaky vasculature of tumours can be exploited to obtain some tumour selectively by making the liposome too large to cross non-leaky vessels. However, large liposomes tend to be cleared as particulates. Thus, some compromise is required and the determining factors will be whether the tumour has abnormally leaky vasculature and to what extend the modified liposomes are spared from the removal system for particulates.

The compositions of the present invention are presented for administration in any conventional pharmaceutically acceptable manner. For instance the compositions may be presented as dry powders, such as lyophilised liposomes, for reconstitution with sterile water or water for injection. Alternatively the compositions may be presented as aqueous dispersions or suspensions ready for injection or as concentrates suitable for dilution, for instance with sterile water or water for injection, so as to form injectable products. The compositions of the invention, when formulated for injection will typically contain conventional diluents or carriers, antioxdants and preservatives, anti-bacterial or anti-microbial agents as well as excipients, formulation aids, buffers, agents to adjust the pH and tonicity of the composition and other conventional auxiliary components used in the art of pharmacy. The compositions of the invention which are presented as dry powders or concentrates for reconstitution or dilution to form injectable products may also contain conventional additives to aid reconstitution or dilution thereof.

The compositions of the invention, where necessary after reconstitution or dilution, are administered to patients in need thereof, for instance patients having or suspected to have solid tumours or dermatological diseases or disorders, in suitable amounts to achieve the necessary therapeutic or diagnostic dose at the target site for the desired duration, without causing clinically unacceptable side effects. The compositions are administered by injection by any conventional route which will afford access to the bloodstream for the multi-molecular structures and associated therapeutic or diagnostic agent. Typically the compositions will be administered by the intravenous, intramuscular or parenteral route. The compositions may be injected in a single dose, as divided doses or by infusion over a period of from several minutes to several hours or even days as appropriate.

The invention will be illustrated with reference to the Figures of the accompanying drawings in which:

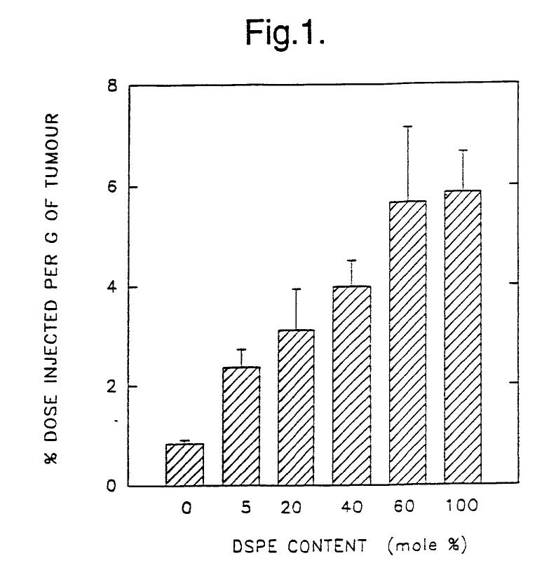

FIG. 1. shows the percent of dose injected per gram of tumour tissue plotted as a bar chart against DSPE content of administered PEGylated liposomes (see Example 4).

FIG. 2 shows the percent of dose injected per gram of liver, spleen and tumour tissue plotted as a bar chart against DSPE content of administered PEGylated liposomes (see Example 4).

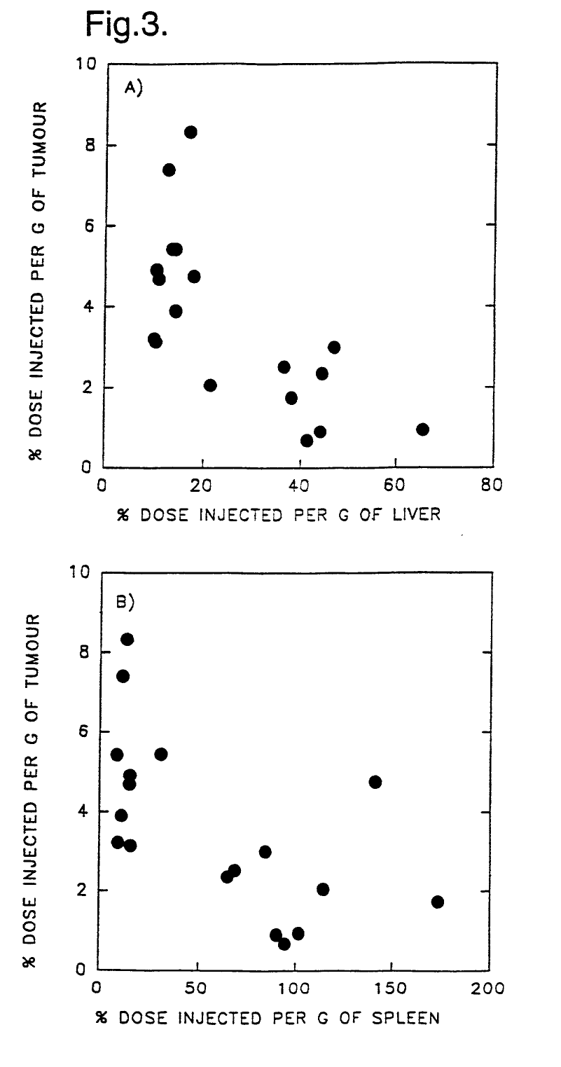

FIG. 3 correlates the percent of dose injected per gram of tumour with the percent of dose injected per gram of liver (a) or spleen (b), (see Example 6).

FIG. 4 shows the percent of ¹¹¹Indium retained plotted as bar charts at 13 (a), 19 (b), 22 (c) and 49 (d) days of storage against DSPE content of liposomes (see Example 6).

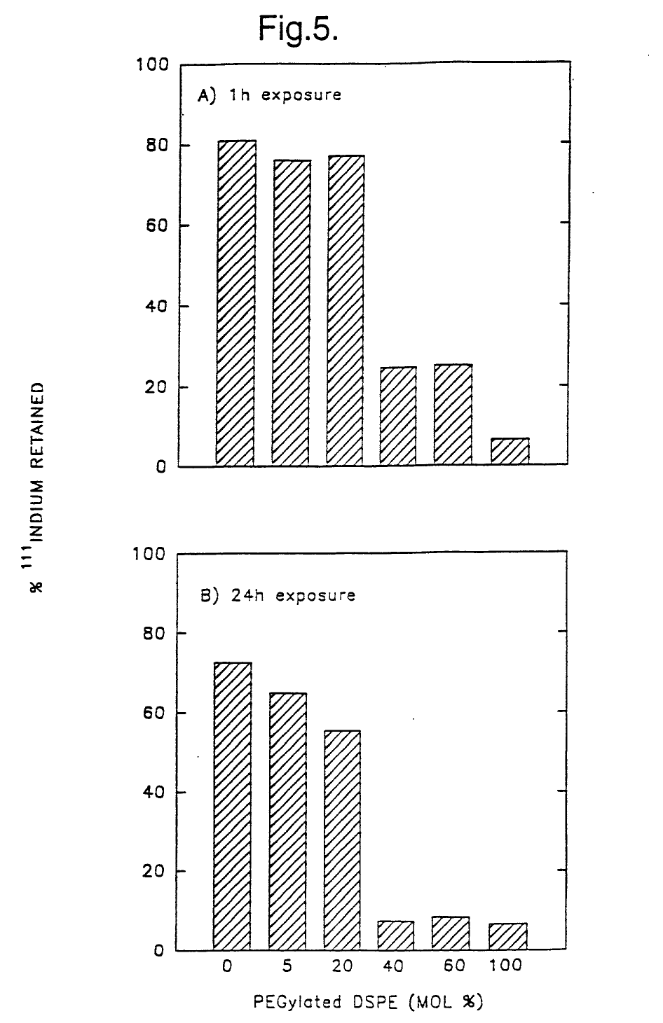

FIG. 5 shows the percent of ¹¹¹Indium retained plotted as bar charts at 1 h and 24 h after exposure to citrated fresh frozen plasma against PEGylated DSPE content of liposomes administered (see Example 6).

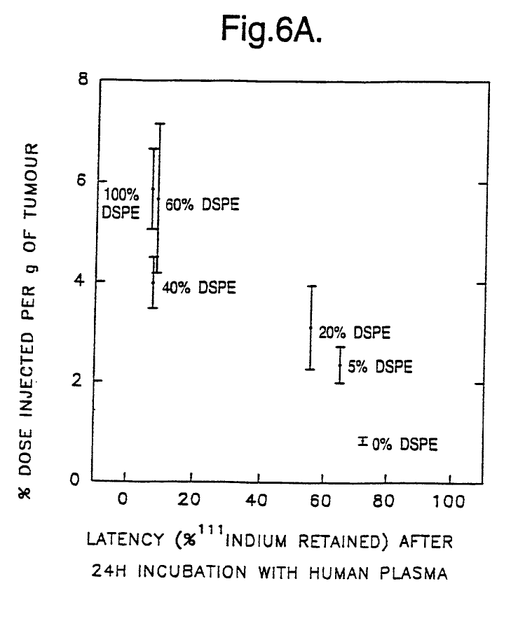

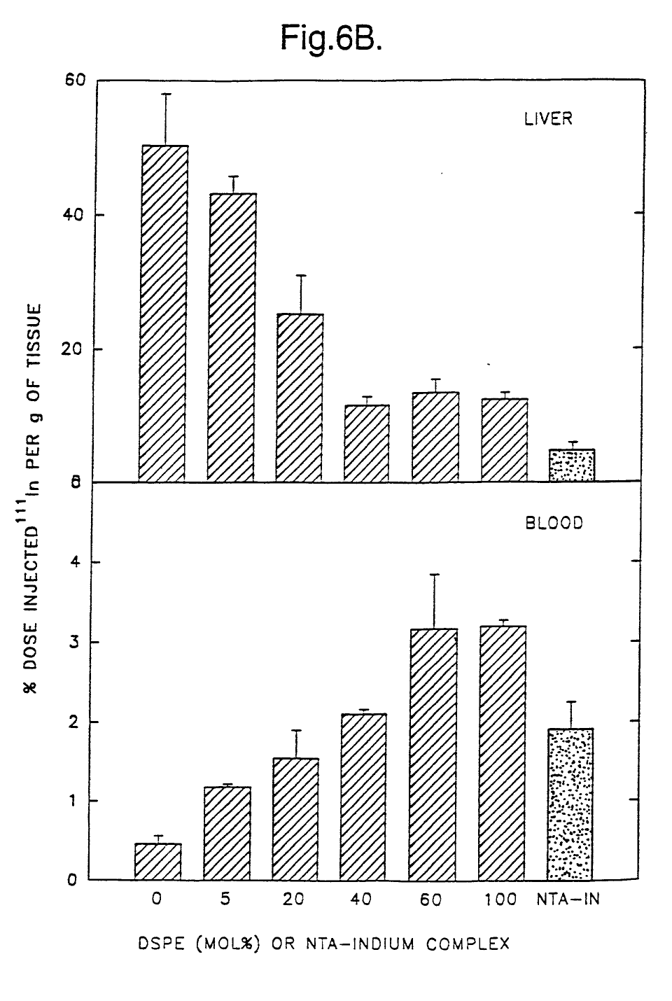

FIG. 6 shows, (a) a plot of the percent dose injected per gram of tumour tissue versus latency (percent ¹¹¹Indium retained) after 24 h incubation with human plasma and, (b), bar caarts of % dose injected of ¹¹¹Indium per gram of liver (upper panel) and blood (lower panel) plotted against DSPE (mol %) for NTA-Indium complex. (see Example 6).

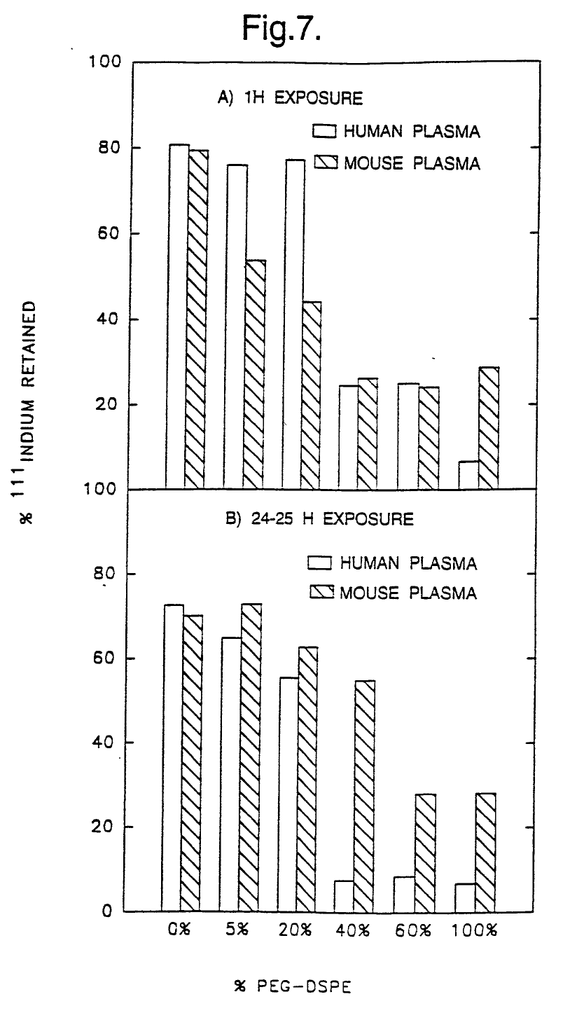

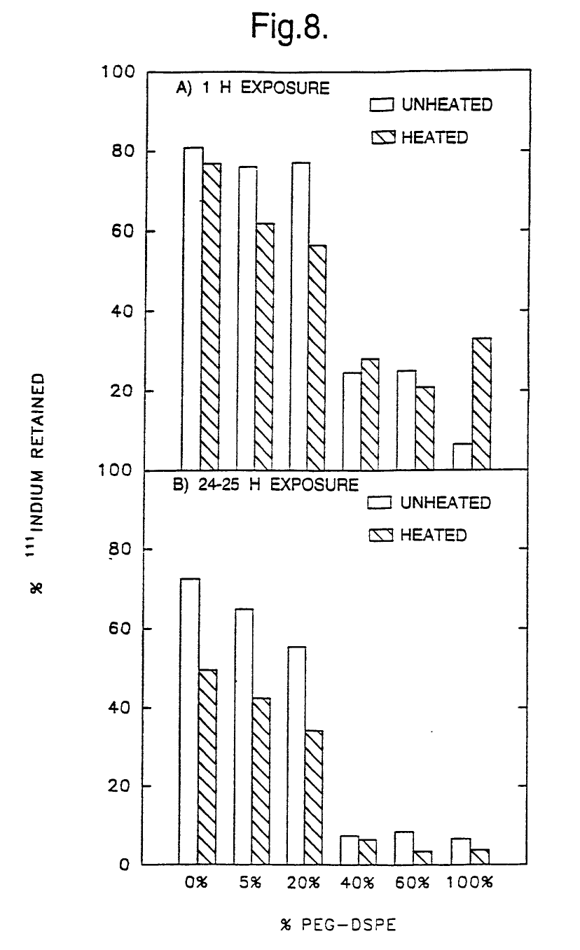

FIG. 7 and 8 each show plots of percent ¹¹¹Indium retained versus percent DSPE in the PEGylated liposomes exposed for either 1 h (A) or 24-25 h (B) to various types of plasma (see Example 6).

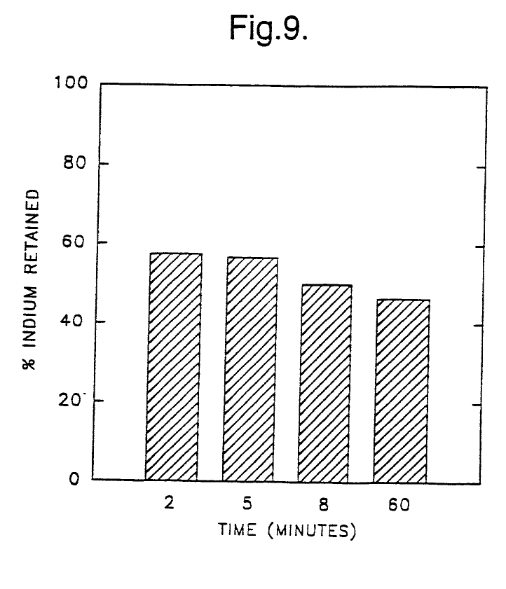

FIG. 9 plots the percent ¹¹¹Indium retained against time (minutes) of exposure to fresh frozen citrated plasma (see Example 6).

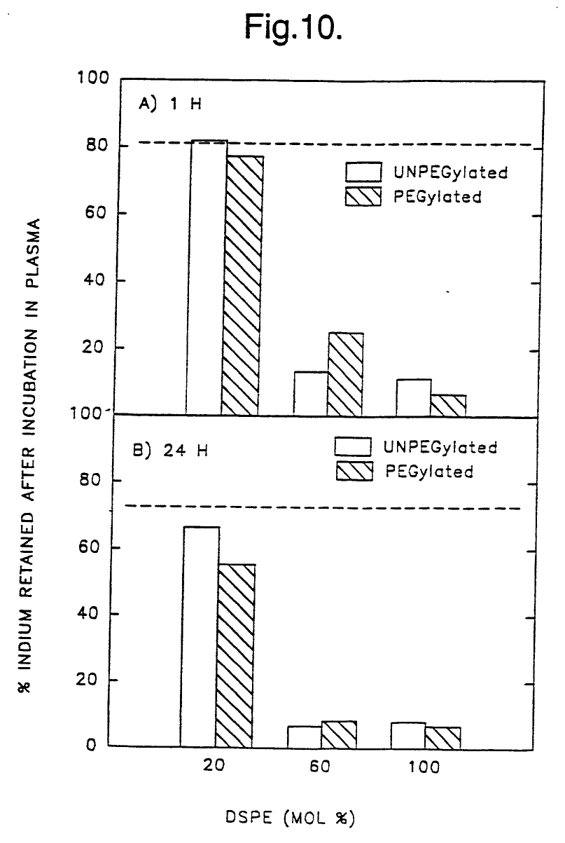

FIG. 10 plots the percent ¹¹¹Indium retained after incubation in plasma for 1 h (A) and 24 h (B) versus DSPE content in unPEGylated and PEGylated liposomes (see Example 6).

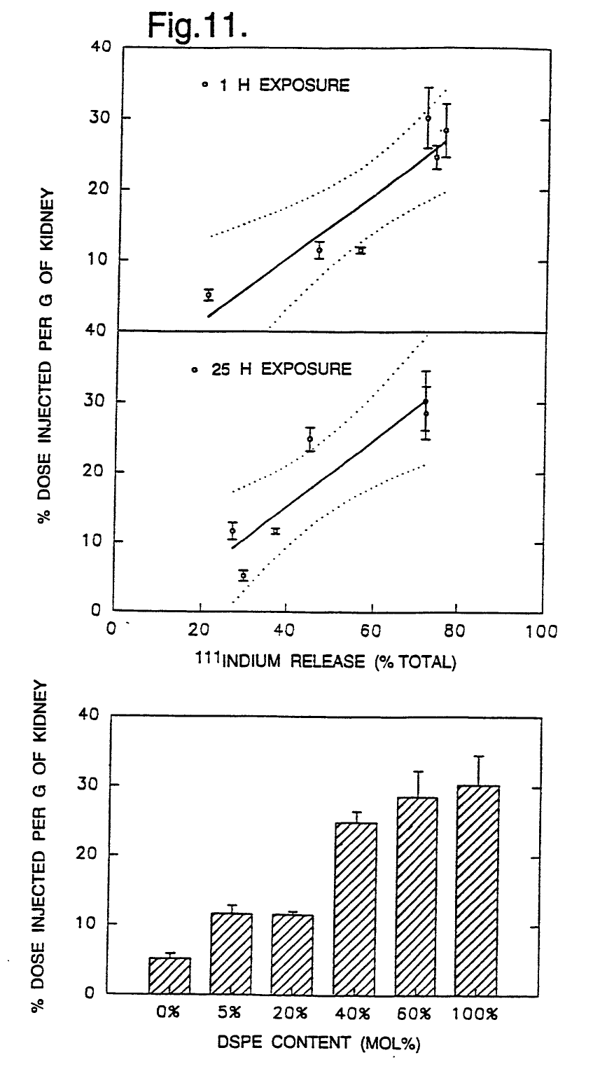

FIG. 11 plots the percent of injected dose per g of kidney tissue at 1 h (upper panel) and 25 h (centre panel) against ¹¹¹Indium released in vitro by exposure to mouse plasma (percent of total) and, as a bar chart, against DSPE content (mol %) of liposomes (bottom panel), (see Example 6).

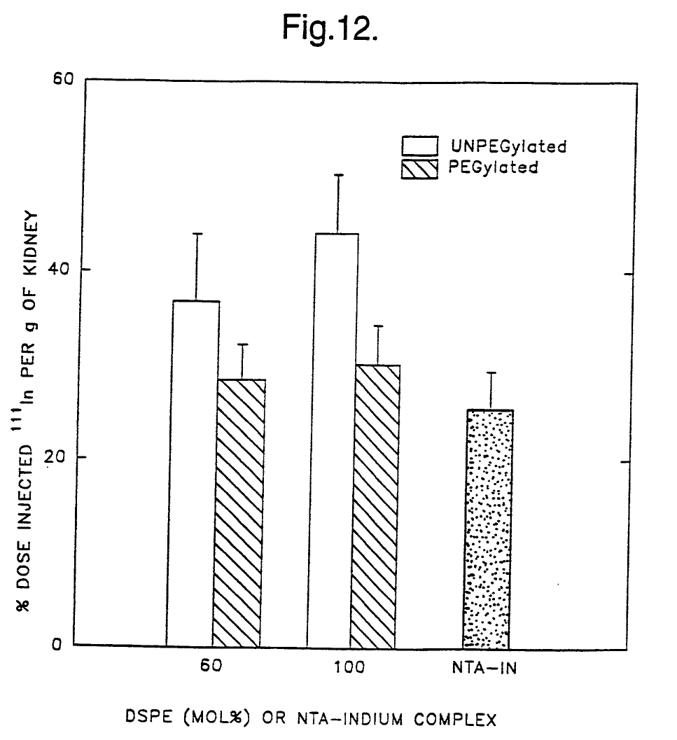

FIG. 12 plots the percent of injected dose of ¹¹¹Indium per gram of kidney against DSPE (mol %) for PEGylated (hatched) and unPEGylated (open) liposomes and Free NTA-Indium complex (see Example 6).

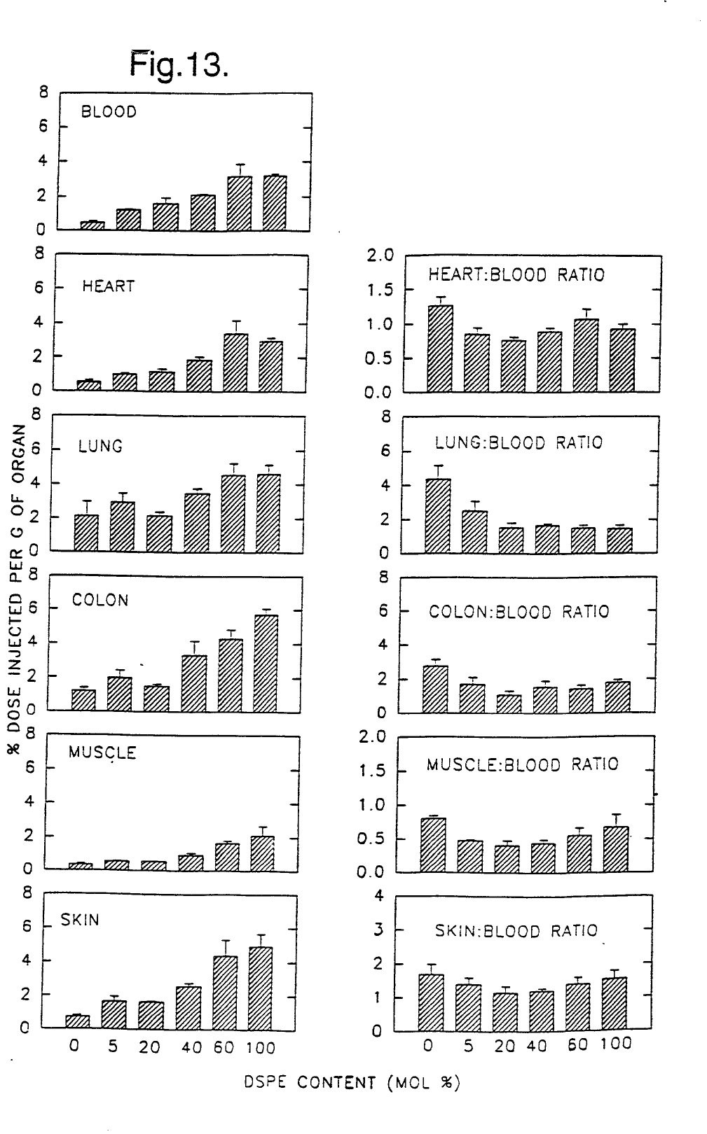

FIG. 13 plots the percent of dose injected per gram of tissue for various organs against DSPE content (mol %) of admnistered PEGylated liposomes (left hand series of graphs) and the organ:blood ratios of those doses against DSPE content (mol %) (right hand series of graphs) (see Example 7).

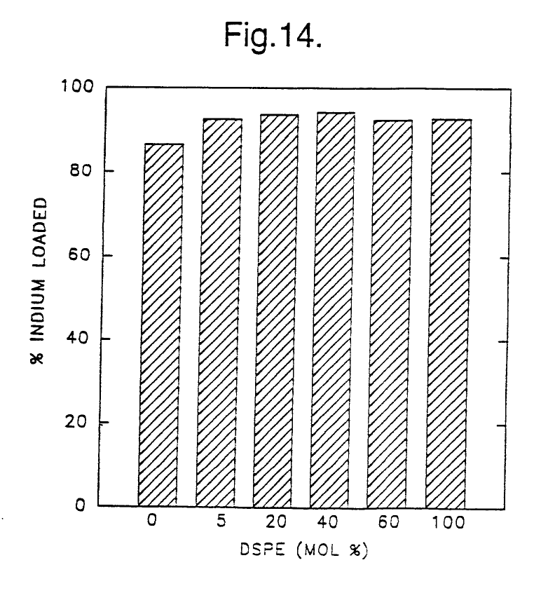

FIG. 14 plots percent of ¹¹¹Indium loaded in liposomes against DSPE content (mol %) of the liposomes (see Example 8).

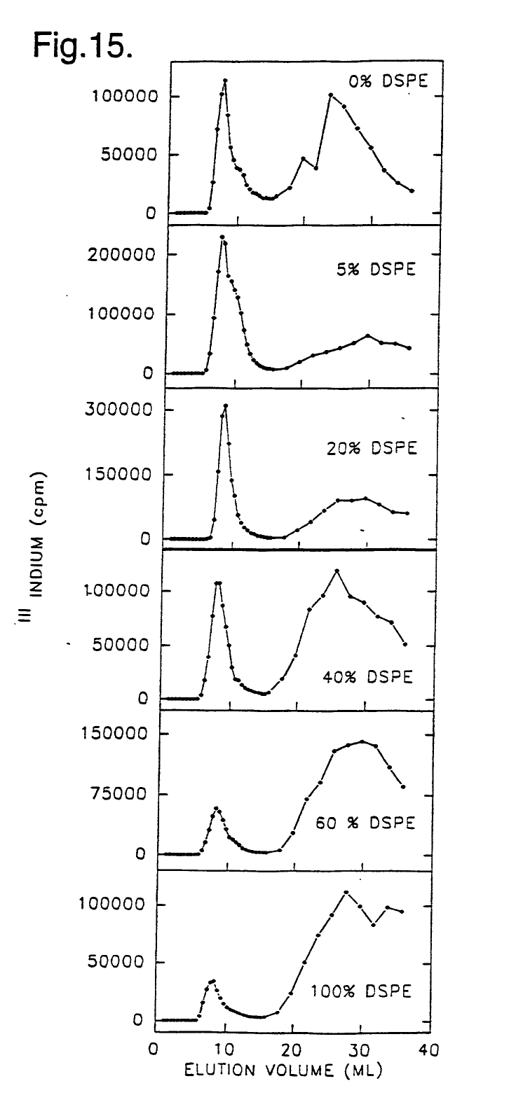

FIG. 15 plots ¹¹¹In counts against elution volume (ml) for various DSPE contents of liposomes (see Example 8).

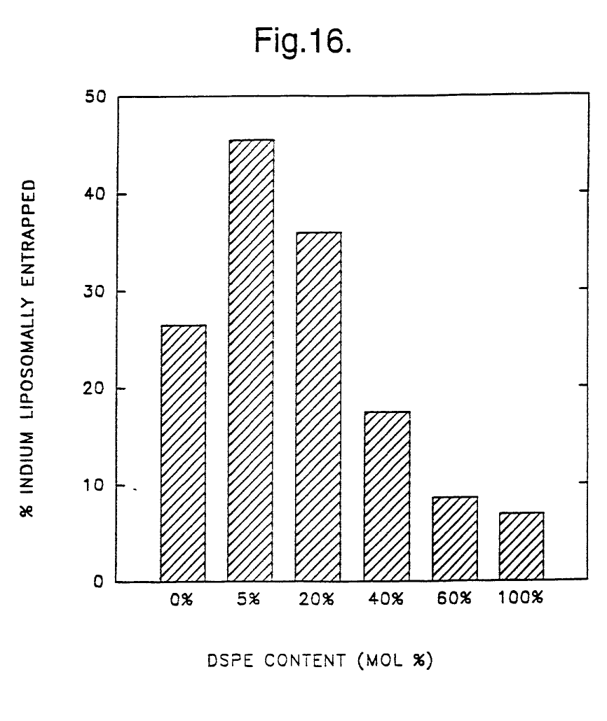

FIG. 16 plots percent of ¹¹¹Indium entrapped in liposomes against the DSPE content (mol %) of the liposomes (see Example 8).

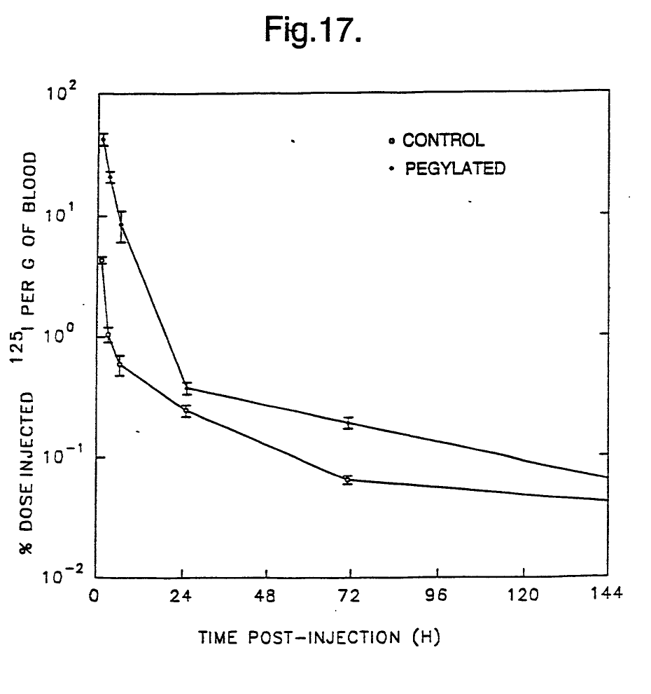

FIG. 17. plots percent of injected dose of ¹²⁵I per gram of blood at various times (h) post injection (see Example 9).

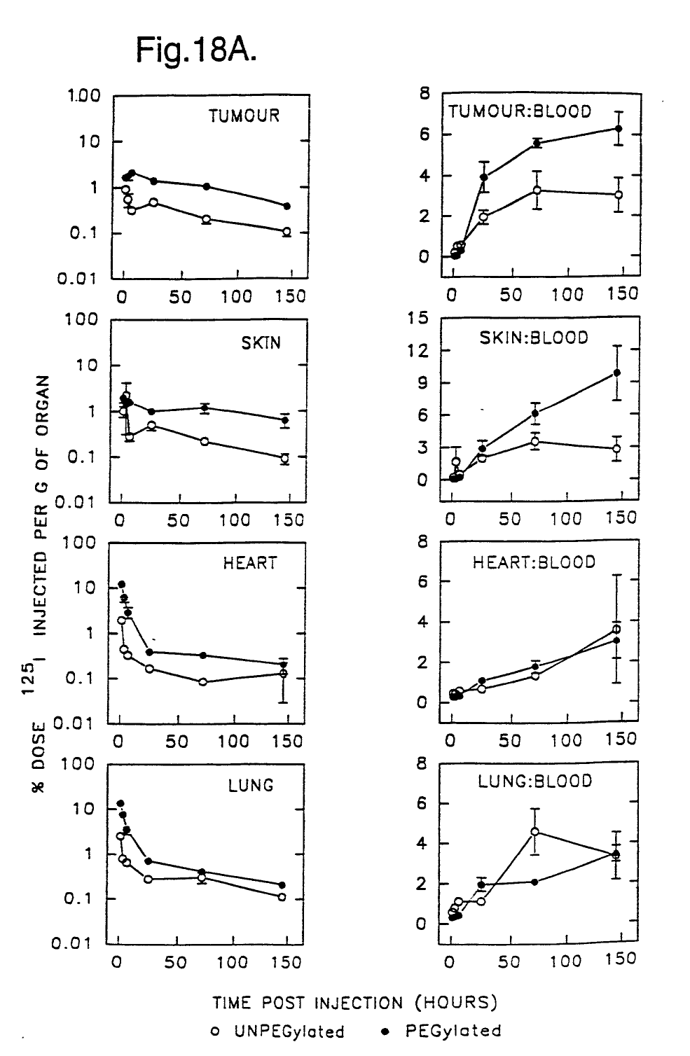

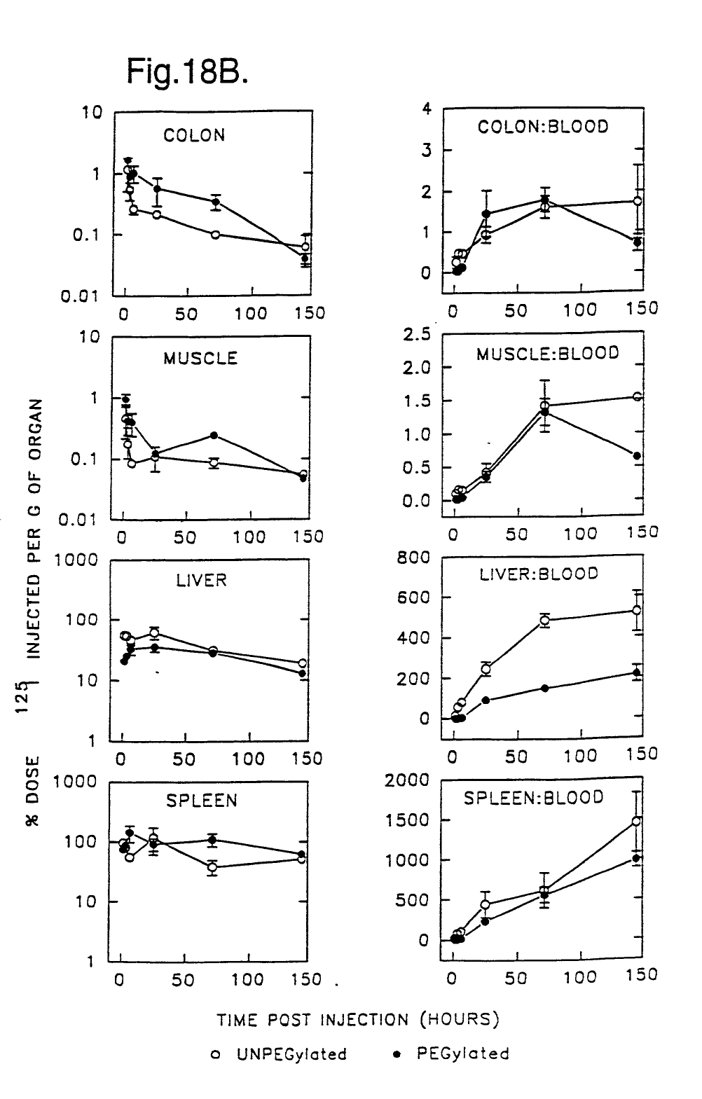

FIG. 18 plots the percent of ¹²⁵I injected per gram of organ for PEGylated (closed circles) and unPEGylated (open circles) liposomes at various times (h) post injection of the liposomes (left hand series of graphs) and the corresponding tissue to blood ratios (right hand series of graphs) (see Example 9).

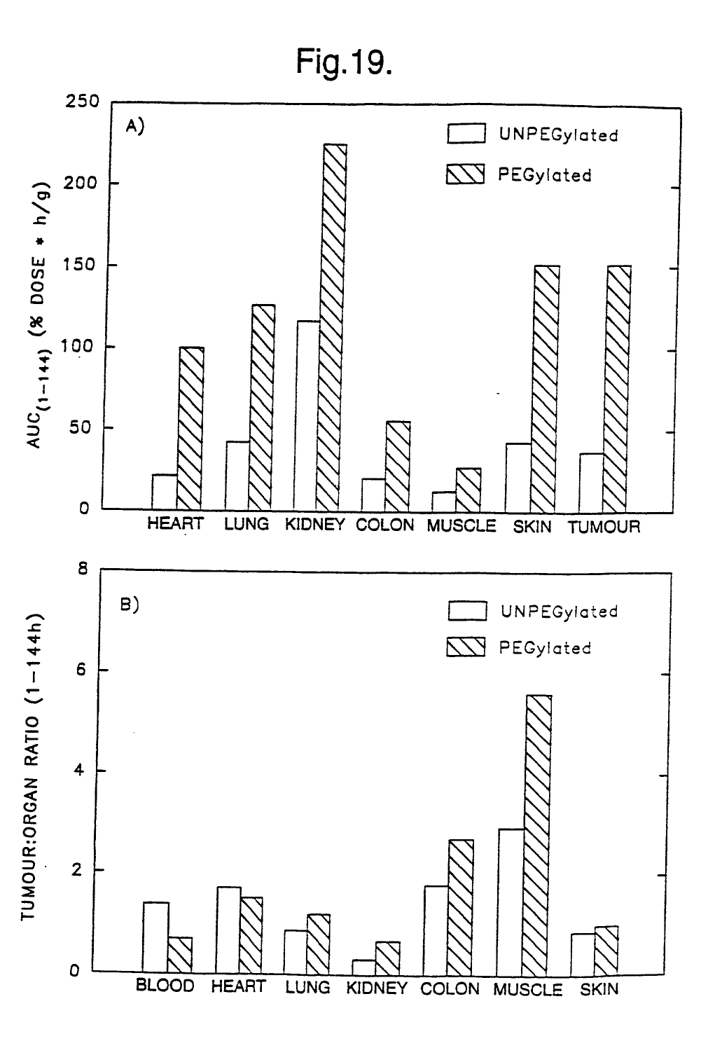

FIG. 19 shows the area under the curve (AUC's) over the period 1 to 144 h taken from the graphs in FIG. 18 for PEGylated (hatched) and unPEGylated (opened) liposomes (upper panel) and the corresponding tumour to organ ratios (lower panel) for the various tissues tested (see Example 9).

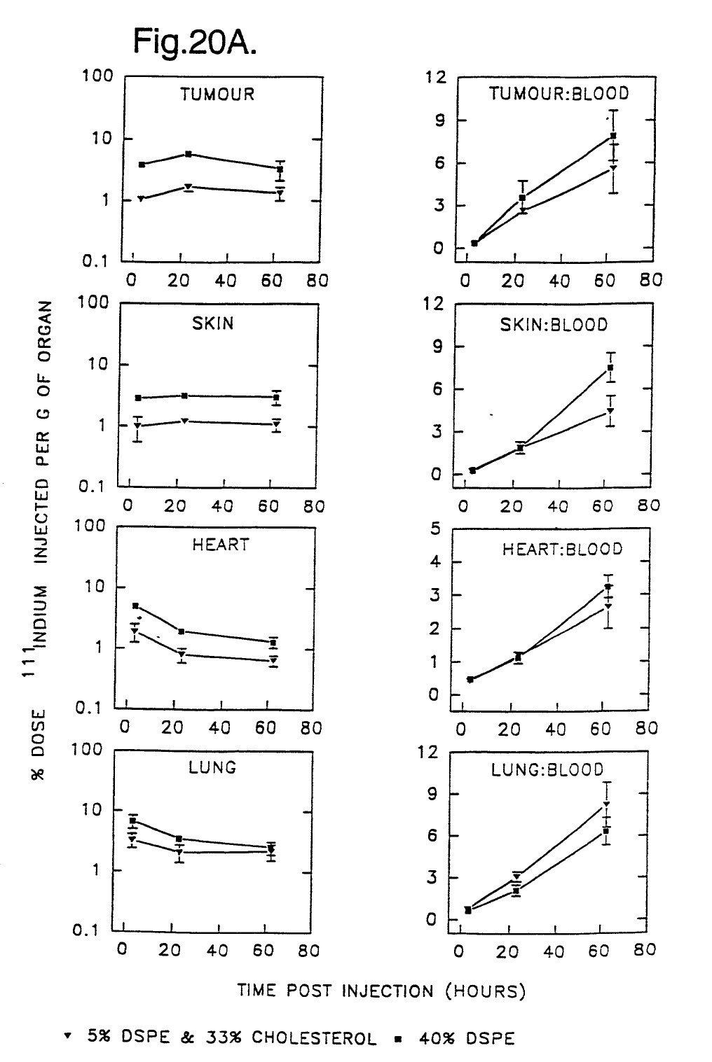

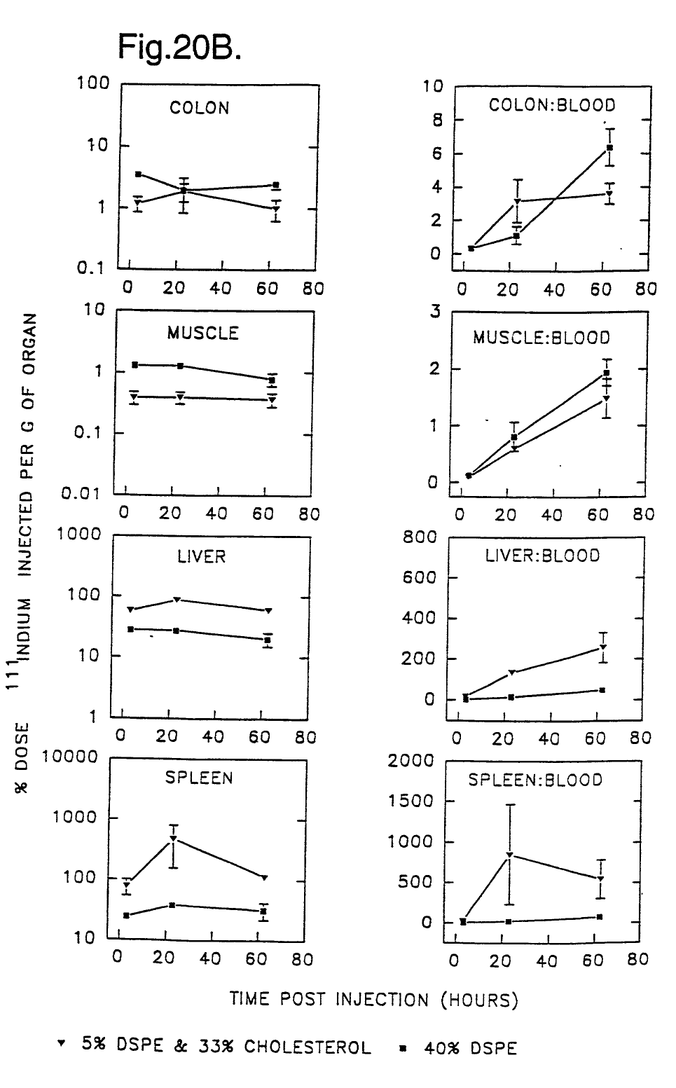

FIG. 20 plots the percent dose of ¹¹¹In per gram of organ against the time (h) post injection of liposomes containing 5% DSPE and 33% cholesterol (triangles) and 40% DSPE without cholesterol (squares) (left hand series of graphs) and corresponding organ:blood ratios (right hand series of graphs) (see Example 9).

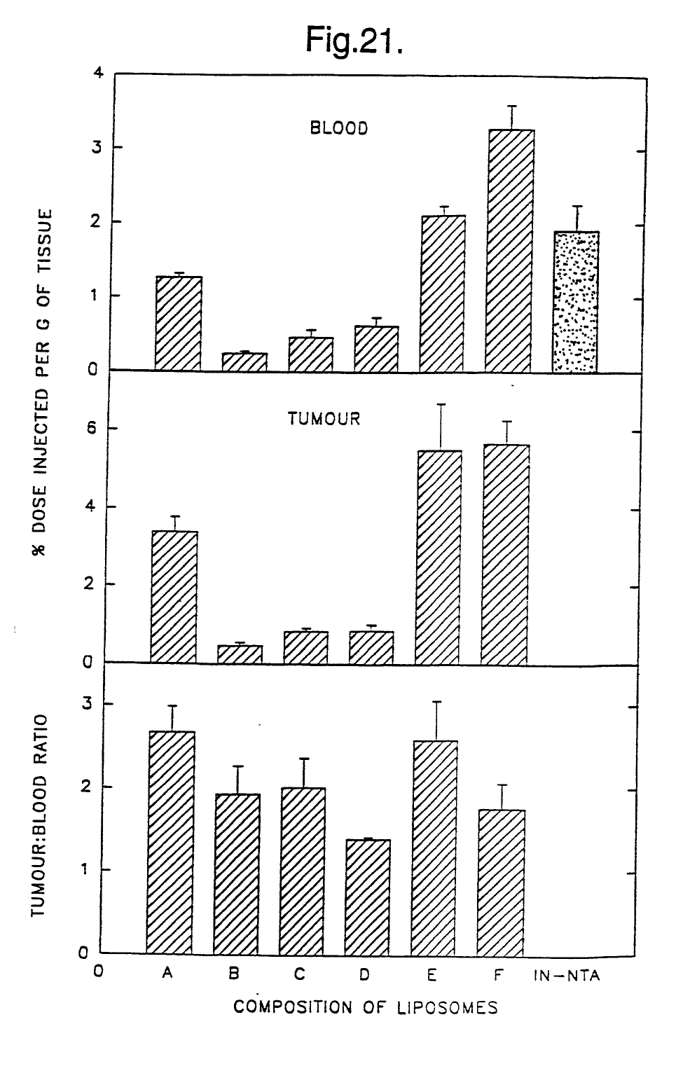

FIG. 21 shows the percent of injected dose per gram of tissue for blood (upper panel) and tumour (lower panel) for various liposome compositions and for Free ¹¹¹In-NTA complexes and gives the corresponding tumour to blood ratios (lower panel), (see Example 10).

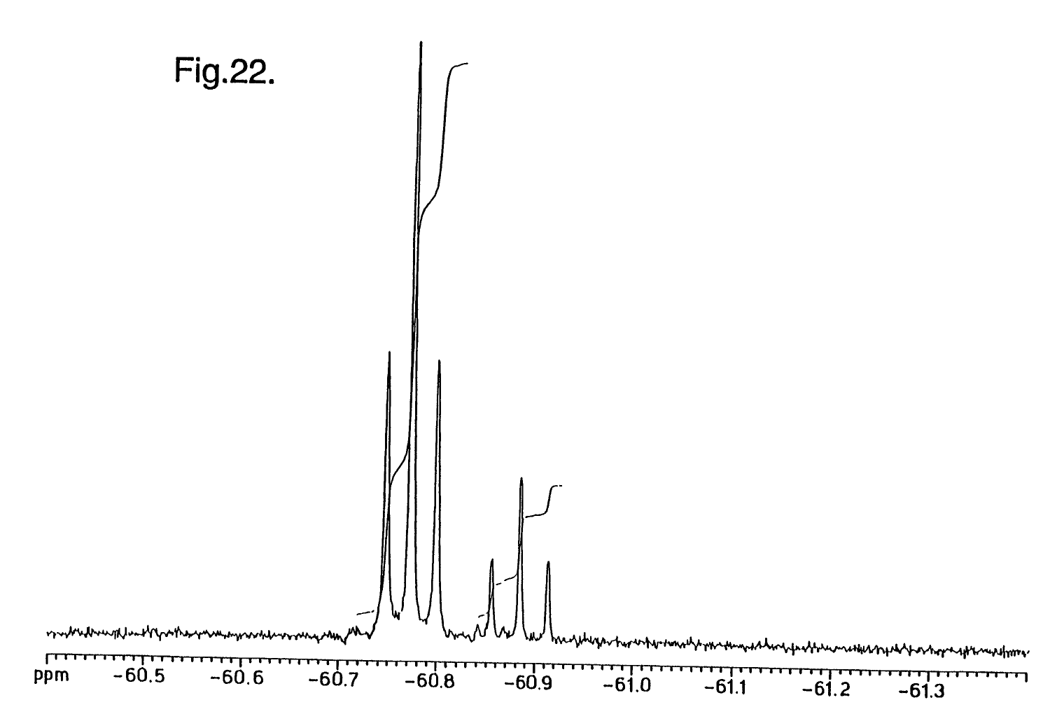

FIG. 22 shows a ¹⁹F-nmr trace of TMPEG-5000 in DMSO (see Example 11).

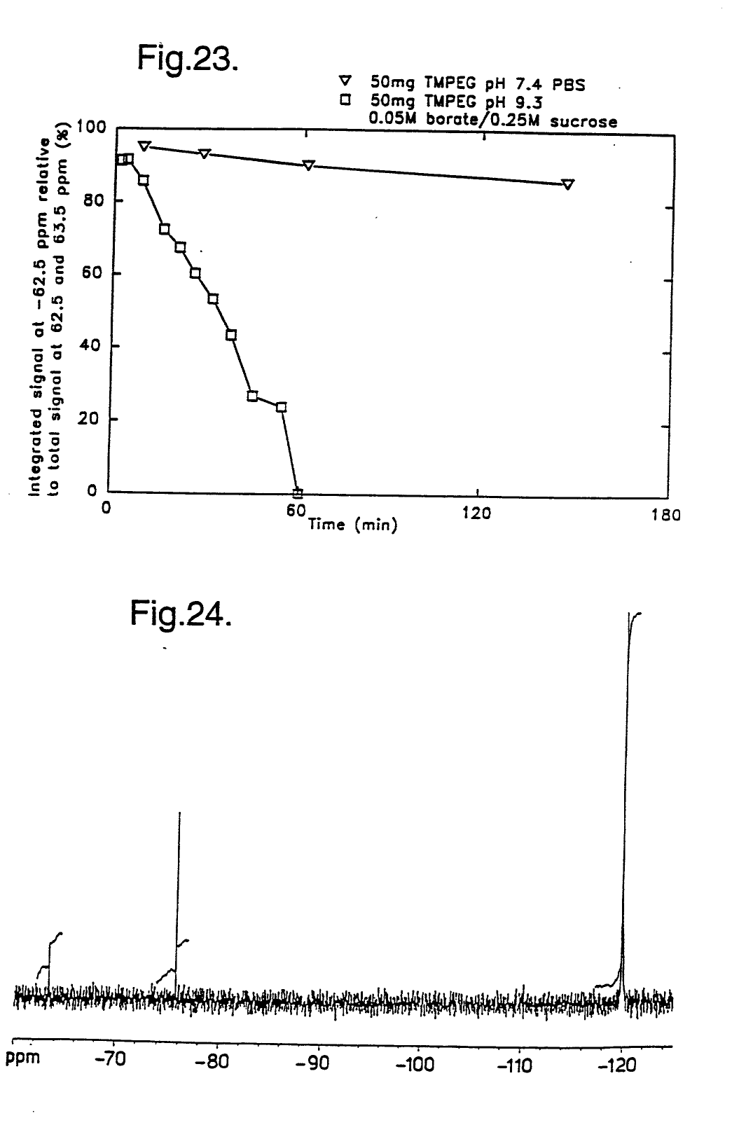

FIG. 23 is a graph of the strength at various times of the ¹⁹F signal at 62.5 ppm (TMPEG-5000) expressed relative to the strength of the two triplets at −62.5 and −63.5 ppm (%) for TMPEG treated with borate buffer (squares) and HEPES buffer (triangles), (see Example 11).

FIG. 24 shows a ¹⁹F-nmr trace of TMPEG-5000 in 50 mM borate pH 9.3 containing 250 mM sucrose after 80 min incubation when all the intact TMPEG had disappeared (see Example 11).

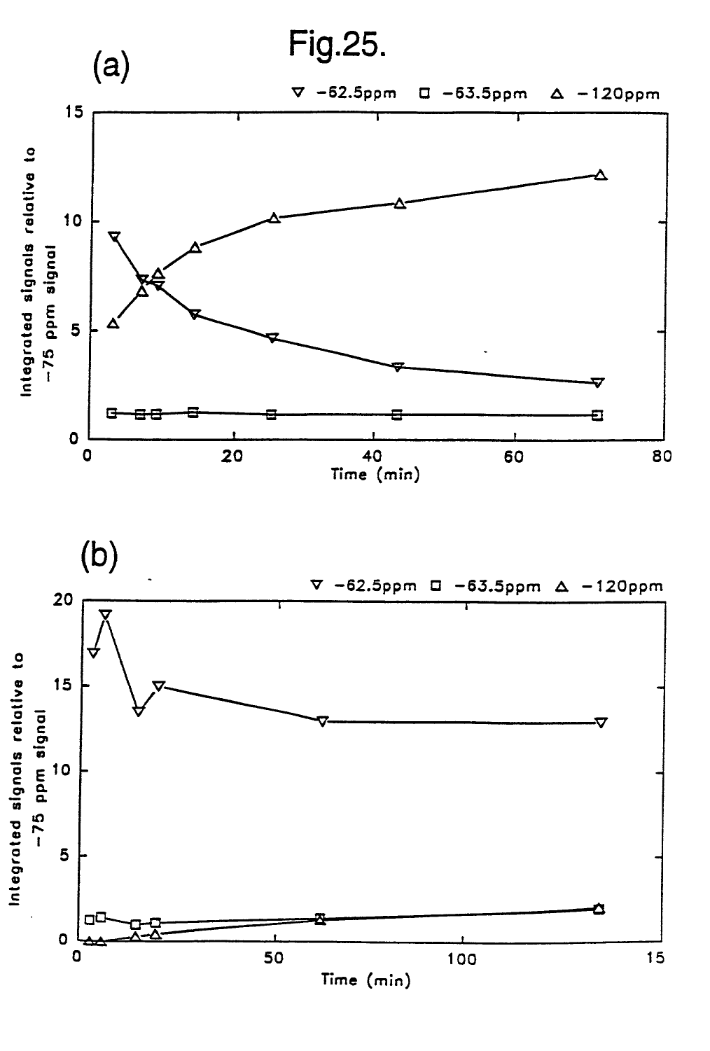

FIG. 25 is a graph of relative signal strength versus time for various species detected by ¹⁹F-nmr when TMPEG-5000 was exposed to a) 50 mM borate pH 9.3 containing 250 mM sucrose and b) 20 mM HEPES pH 7.4 containing 290 mM sucrose (see Example 11).

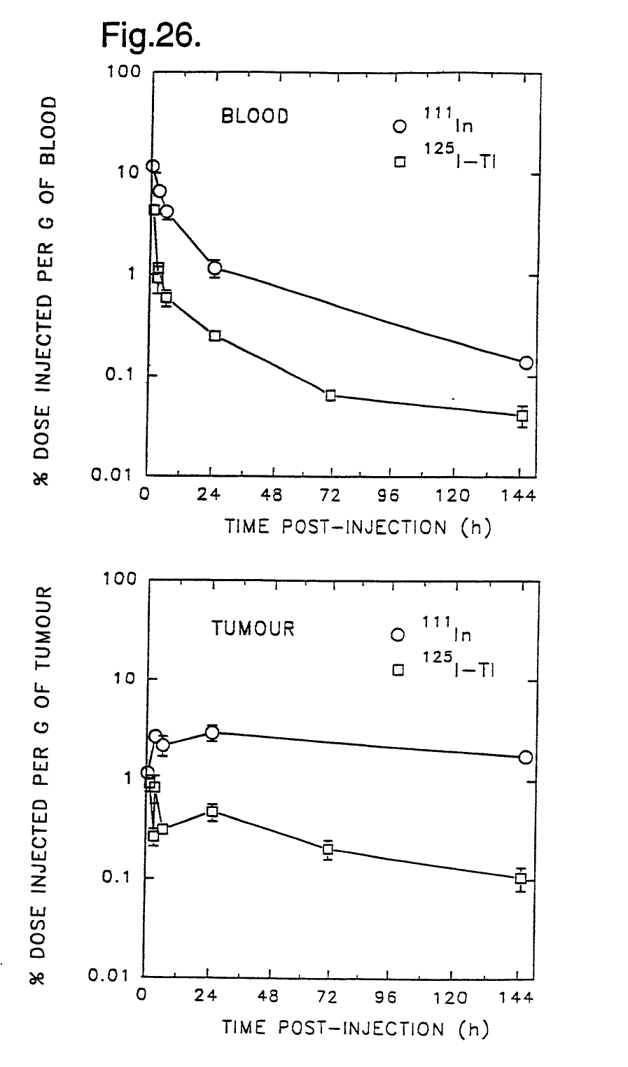

FIG. 26 shcws the percent of injected dose per gram of tissue for blood (upper panel) and tumour (lower panel) plotted against time post-injection (h) for ¹¹¹In (circles) and ¹²⁵I-TI (squares) administered in liposomes.

### EXAMPLES

### Example 1

### Preparation of PEG-Modified DSPC/DSPE Liposomes Via Exterior PEGylation of Incorporated Phosphatidyl Ethanolamine

The liposomes were typically prepared by extrusion of a 10 mg/ml liposomal suspension. To produce 5 ml of 10 mg/ml phospholipid suspension, lipid films with a total content of 50 mg phospholipid (PL) were prepared by mixing quantities (shown in Table 1, μl) of DSPC (at a concentration of 100 mg/ml in chloroform) and DSPE (at a concentration of 100 mg/ml in chloroform/methanol, 2:1) as a summarised in Table 1.

TABLE 1

| | DSPC to DSPE mol % | | | | | | |
|---|---|---|---|---|---|---|---|
| | 100:0 | 95:5 | 80:20 | 70:30 | 60:40 | 40:60 | 0:100 |
| DSPC | 500 | 476 | 404 | 356 | 307 | 207 | 0 |
| 100 mg/ml | | | | | | | |
| DSPE | 0 | 24 | 96 | 144 | 193 | 293 | 500 |
| 100 mg/ml | | | | | | | |

| | |
|---|---|
| Ionophore | 109 μl to each tube |
| 0.5 mg/ml | |
| ³H Cholesterol | 25 μCi to each preparation |
| Hexadecylether | |

Ionophore A23817 was incorporated into the lipid bilayer at a molar concentration of 0.1 μmol per 50 mg total phospholipid (54.4 μg per 50.0 mg phospholipid). ³H Cholesterol Hexadecylether (25 μCi), a non-exchangable lipid marker, was added in selected experiments to aid following the lipid concentration throughout procedures.

To produce the thin lipid film, the solvent was evaporated carefully. This can be done in many different ways, for example evaporation by blowing nitrogen gas as follows: the outlet was placed 5 cm above the surface of the solvent and the nitrogen flow was adjusted to avoid bubbling at the solvent/air interface. The pressure of nitrogen has to be adjusted according to the number of outlets.

Although most of the solvent was removed by nitrogen gas, it is important to remove any residual traces of the solvents in order to get a good liposomal preparation. Therefore it is imperative to place the dried film in a desiccator overnight under vacuum, to remove any traces of solvent.

To disperse the lipids, 5 ml of the buffer containing the water soluble component(s) to be entrapped (for instance a radiolabelled or fluorescent compound or nitrilotriacetic acid, NTA, for subsequent loading of ¹¹¹Indium) were added to the lipid film at room temperature. The mixtures were then placed in an orbital shaker with gentle shaking for 20 h at room temperature. After the 20 h swelling, the mixtures were subjected to several cycles of warming up to 65° C. (2 min) and vortexing (1 min) until complete dispersion of the lipid.

The liposomal suspension was then subjected to 5 cycles of freezing and thawing by immerising the tube in liquid N₂ for 1-2 min (or the time required for the liposomal preparation to be frozen) followed by immersion in water at 65° C. for 1-2 min (or the time required to have a liquid liposomal suspension).

The liposomal suspension was then extruded at 65° C. (temperature provided by a thermobarrel connected to a recirculating water bath) through polycarbonate filters (double stack filters) as follows: through 0.4 micron filters 5 times; through 0.2 micron 5 times and through 0.1 micron 10 times. This produces liposomes of average size circa 100 mm.

The liposomes with entrapped contents were then separated from the unentrapped water soluble components by exchanging the buffer, e.g. by gel permeation chromatography. Commercial PD-10 columns (Sephadex G-25, fractionation range 1000-5000 KDa) were found to be suitable. Using these columns, liposomes were collected with the void volume and NTA (or other water soluble contents) with the total volume of the column. The PD-10 column was equilibrated with the appropriate buffer and loaded with 2 ml (maximum) of the extruded liposomal suspension. After collecting fraction 1, fractions 2 to 30 were eluted with 300 μl buffer. The location of the liposomes was established by quantifying the lipid label (i.e. ³H) by scintillation counting. Fractions containing liposomes were then pooled and lipid content established by the ³H content and also by estimation of phosphorus.

The liposomes can then be loaded with ¹¹¹ In (if they had been produced in the presence of for instance NTA) by incubation with ¹¹¹In (¹¹¹Indium hydrochloride formulated in 0.04 M HCl) at a ratio of for example 0.8 mCi per 10 mg of phospholipid.

The extruded liposomes loaded with the appropriate contents were then PEGylated by reaction with TMPEG for 2 h at room temperature in an appropriate buffer (the advantages and disadvantages of different buffers are discussed further below, examples include:

1) 50 mM borate buffer pH 9.3 containing sucrose 250 mM;

2) 50 mM phosphate buffer pH 7.4 also containing 250 mM sucrose and

3) 20 mM HEPES 145 mM NaCl pH 7.4).

The liposomal suspension was adjusted to a final phopholipid concentration of 2 mg/ml in a reactin mixture containing TMPEG 166 mg/ml (alternative strategies are discussed below).

The PEGylated liposomes were collected free of unreacted TMPEG by gel permeation chromatography using Sepharose CL-4B. The maximum loading for a Sepharose column with dimensions 7 cm high, 2.4 cm diameter, is 1 ml of liposomal suspension of circa 2 mg/ml total phospholipid and TMPEG 166 mg/ml. The column was first equilibrated with the appropriate buffer (for instance a buffer suitable for injection). The excess buffer was drained from the top of the column before loading the liposomes. After collecting fraction 1, fractions 2 to 30 were eluted with 500 μl buffer and fractions 31 to 40 with 2 ml of buffer. PEGylated liposomes were collected with the void volume.

For DSPE:DSPC:CHOL (5:62:33 mol %) liposomes, the method above was followed with the following exceptions:

1) that orbital shaking was omitted and rigourous vortexing used instead (with liposomes containing higher DSPE content and no cholesterol this procedure produced frothing and orbital shaking and a longer time was therefore substituted);

2) the liposomes were constructed using 50 mM phosphate pH 7.4 containing 250 mM sucrose and NTA;

3) The buffer used for the chromatographic separation of NTA was 50 mM phosphate pH 7.4 containing 250 mM sucrose;

4) Before indium loading liposomes were filtered through 0.2μ filter (Acrodisc 13) to remove any aggregates;

5) The ¹¹¹Indium loaded was 0.5 mCi per 10 mg phospholipid;

6) the final concentration of phospholipid in the PEGylation reaction was 3.5 mg/ml containing TMPEG at 150 mg/ml.

The extent of PEGylation was monitored by thin layer chromatography of phospholipids and phosphorous estimation of the DSPC, DSPE and PEG-DSPE. Where 166 mg/ml TMPEG was used at pH 9.3 there was substantial leakage of contents when the pH was changed to 7.4. Since PEGylated liposomes with high DSPE content (60 and 100 mol %) withstood this pH shift better than unPEGylated liposomes (9.3 versus 2.6% and 7.4 versus 1.7% retained contents respectively), liposomes retaining their contents could have been enriched for heavily PEGylated liposomes. This complicates accurate assessment of the mol % of PEG-lipid, since lipid from both “empty” and “loaded” liposomes is assessed in TLC. In addition, high TMPEG concentrations may cause aggregation of liposomes (via volume exclusion effects) and hence impede equal access of TMPEG to all the liposomes. If this were performed at pH 7.4 (i.e. omitting the lysis inducing step of a pH change) a preparation might have heterogeneous PEGylation, i.e. contain a mixture of heavily PEGylated and un/lightly PEGylated lipcsomes. In addition to these considerations, high PEG concentrations are known to render the lipid bilayer permeable to PEG. This would allow some PEGylation of the interior, this possibility should therefore be taken into account if applying a method to assess PEGylation which measures total lipid. We have developed a method based on the Childs assay for PEG that allows exterior and total PEG to be assayed. Since it is the exterior PEG that provides the barrier to RES uptake, it is important to compare liposomes with respect to their exterior PEG content. Given the potential problems of high TMPEG concentrations (aggregation, bilayer transfer during PEGylation, fusion and, with susceptible liposomes transition to non-liposomal structures), an alternative and possibly preferable PEGylation scheme is to add TMPEG in a step-wise fashion so that it is at sub-aggregation, sub-fusogenic doses during the early stages of the reaction. If necessary, excess TMPEG can be removed between addition of aliquots of TMPEG.

### Example 2

### Preparation of PEG-Modified DOPC/DOPE Liposomes Via Exterior PEGylation of Incorporated Phosphatidyl Ethanolamine

The liposomes were typically prepared by extrusion of a 10 mg/ml liposomal suspension. To produce 4 ml of 10 mg/ml phospholipid suspension, lipid films with a total content of 40 mg phospholipid (PL) were prepared by mixing DOPC (at a concentration of 20 mg/ml in chloroform) and DOPE (at a concentration of 10 mg/ml in chloroform/methanol, 2:1). The lipid film was produced as in Example 1.

To disperse the lipids, 4 ml of the buffer containing the water soluble component(s) to be entrapped (e.g. ¹²⁵I-tyraminylinulin, TI) were added to the lipid film at room temperature. The mixtures were then vortexed vigorously. The mixtures were subjected to several cycles of warming up to 65° C. (2 min) and vortexing (1 min) until complete dispersion of the lipid. The liposomal suspension was then subjected to 5 cycles of freezing and thawing by immersing the tube in liquid N₂ for 1-2 min (or the time required for the liposomal preparation to be frozen) followed by immersion in water at 65° C. for 1-2 min (or the time required to have a liquid liposomal suspension).

The liposomal suspension was then extruded at circa 65° C. through polycarbonate filters (double stack filters) as follows: through 0.4 micron filters 5 times; through 0.2 micron 5 times and through 0.1 micron 10 times. This produces liposomes of average size circa 100 nm.

The liposomes with entrapped contents were then separated from the unentrapped water soluble components using 10 ml syringe barrel Sepharose CL-4B column using 20 mM HEPES 145 mM NaCl buffer pH 7.4 Liposomes were collected with the void volume and TI (or other water soluble contents) with the total volume of the column. The column was equilibrated with the same buffer and loaded with 1 ml of the extruded liposomal suspension. After collecting fraction 1, fractions 2 to 49 were eluted with 400 μl buffer. The location of the liposomes was established by quantifying the ¹²⁵-TI (or other label) by gama counting. Fractions containing liposomes were then pooled.

The extruded liposomes loaded with the appropriate contents were then PEGylated by reaction with TMPEG for 2 h at room temperature in 20 mM HEPES 145 mM NaCl pH 7.4. The liposamal suspension was adjusted to a final phospholipid concentration of 3.4 mg/ml in a reaction mixture containing TMPEG 70 mg/ml.

### Example 3

### Preparation of PEG-Derivatised Phosphatidyl Ethanolamine for Incorporation into Liposomes

In principle PEG-liposomes should have an essentially identical biological distribution, irrespective of whether they are prepared by incorporation of PEG-lipid or PEGylation of the exterior of the liposome. The body's clearance mechanisms “see” only the exterior PEG, thus a liposome containing 10 mol % PEG-DSPE is equivalent to a liposome containing 10 mol % DSPE which has been fully PEGylated on the exterior, even though PEG-DSPE in the latter case constitutes only 5 mol % PEG-DSPE with respect to total lipid. One difference will be that the carrying capacity will be reduced by PEG-DSPE to an extent determined by the interior PEG-chain content and the molecular size of the content (PEG is known to exclude proteins from surfaces). Heavy PEGylation will lose significant proportions of the liposomal cavity. This should not however affect the biological behaviour. The two methods both have advantages and disadvantages:

### Exterior PEGylation

1) Does not waste interior capacity

2) Can readily achieve very high levels of PEGylation

3) Leads to formation of aggregates if the activated PEG concentration is high.

### PEGylation Via Incorporation

1) Wastes interior space

2) Encounters problems of micelle formation and foaming when high PEG-DSPE levels are used [Allen et al, (1991)]

3) Fails to incorporate all the added PEG lipids [Allen et al, (1991)]

4) With some PEG lipids [Allen et al, (1991)], but not others [Blume and Cevc, (1993)] imposes a relatively low limit (circa 7-10 mol %) onto the amount of PEG-lipid which can be incorporated.

Thus the selection of which production protocol to follow is somewhat arbitrary and depends on the requirements deemed important for a particular application.

In view of the controversy over whether very highly PEGylated liposomes can be prepared by the incorporation route, it is not necessarily possible to achieve comparable results with tumour and skin retention to those observed in the examples below. However, Cevc claims to have made liposomes with up to 100 mol % PEG-DSPE, (i.e. via incorporation of pre-formed PEG-lipid) thus it is not the intention of the inventors to limit the invention to exterior PEGylation.

The production of PEG-lipid is well established; a suitable scheme is as follows:

Distearoylphosphatidylethanolamine (20 mg, 27 μmol) was dissolved in 3 ml of dry chloroform/dry methanol (5/2 vol/vol) with slight warming. Tresyl-monomethoxy PEG-5000 (150 mg, 27 μmol) dissolved in 1 ml of dry chloroform/dry methanol (5/2) was added. The mixture was stirred at 50° C. in the presence of sodium carbonate (290 mg) until the ninhydrin positive DSPE spot detectable on thin layer chromatography disappeared. After removing the sodium carbonate by centrifugation, the MPEG derivative was precipitated from dry diethyl ether and dried under reduced pressure. The purity of the sample was determined by ¹H nmr using the relative integrals of the methylene resonance of the CH₂CH₂O units of the PEG chain and the methylene resonance of the stearoyl chain of the phospholipid.

### Example 4 and Comparative Examples

### Influence of PEG-Modification on Tumour Concentration & Tumour to Blood Ratios

Table 2 gives typical results. With the exception of PEGylated DSPE-DSPC:chol liposomes with only 5 mol % DSPE, in all other cases the PEGylated liposomes showed enhanced tumour concentration over unPEGylated controls (either controls lacking the PEG-lipid or containing the derivatisable lipid in unPEGylated form). Tumour to blood ratios were substantially above 1 and either increased or were maintained with respect to unPEGylated controls. Where the degree of substitution by PEG was compared directly in a liposome of simple composition (DSPC:DSPE) there was a trend of improving tumour concentration of liposomally administered contents with increasing PEGylated DSPE content (FIG. 1).

In contrast to these results, the results of others show declining tumour to blood ratios with PEGylation. Furthermore, all previously published PEGylated examples (i.e. including those for which unPEGylated controls were not given) showed substantially lower tumour to blood ratios than the examples of the present invention given in table 2. It should be noted that the major difference between the two sets of results lies in the extent of PEGylation. In the published examples the mol % of PEG-PE was 7.1 mol %, 5.0 mol %, 6.25 mol % and 6.25 mol % in the examples (from top to bottom table 3). In one of the two examples of Table 2 with 5% PEGylated DSPE, the tumour to blood ratio also fell with respect to the unPEGylated control, whereas in all other examples (all except 1 of which had PEGylated DSPE at = or >20 mol %), the tumour to blood ratios either rose, or remained to same with PEGylation.

TABLE 2
THE INFLUENCE OF PEG-MODIFICATION ON TUMOUR ENTRAPMENT

| Lipid | | | | | | | | | | |
|---|---|---|---|---|---|---|---|---|---|---|
| composition | | Blood¹ | | | Tumour¹ | | | Tumour to Blood² | | |
| (mol %) | PEG | 22-25 h | 60-72 h | 110-145 h | 22-25 h | 60-72 h | 110-145 h | 22-25 h | 60-72 h | 110-145 h |
| (contents) | # | (±SEM) | (±SEM) | (±SEM) | (±SEM) | (±SEM) | (±SEM) | (±SEM) | (±SEM) | (±SEM) |
| [¹¹¹Indium-NTA] | | | | | | | | | | |
| DSPE:DSPC:CHOL | + | 0.66 | 0.29 | 0.095 | 1.70 | 1.32 | 0.82 | 2.63 | 5.57 | 8.63 |
| (5:62:33) | | (±0.134) | (±101) | (±0.004) | (±0.272) | (±0.326) | (±0.043) | (±0.266) | (±1.704) | (±0.727) |
| DSPE:DSPC:CHOL | − | 1.26 | 0.33 | 0.21 | 3.38 | 2.04 | 1.78 | 2.68 | 6.21 | 8.96 |
| (5:62:33) | | (±0.057) | (±0.052) | (±0.029) | (±0.392) | (±0.325) | (±0.227) | (±0.308) | (±0.738) | (±2.047) |
| DSPE:DSPC | + | 1.90 | 0.42 | − | 5.80 | 3.26 | − | 3.604 | 7.905 | − |
| (40:60) | | (±0.446) | (±0.107) | − | (±0.074) | (±1.166) | − | (±1.155) | (±1.765) | − |
| [¹¹¹I-Tyraminylinulin | | | | | | | | | | |
| DOPE:DOPC | + | 0.38* | 0.19 | 0.063* | 1.39* | 1.06 | 0.38* | 3.89* | 5.54 | 6.27* |
| (21:79) | | (±0.044) | (±0.021) | (±0.007) | (±0.166) | (±0.123) | (±0.041) | (±0.746) | (±0.225) | (±0.812) |
| DOPE:DOPC | − | 0.25 | 0.065 | 0.041* | 0.47 | 0.20 | 0.11* | 1.93 | 3.26 | 2.99* |
| (21:79) | | (±0.0283) | (±0.005) | (±0.009) | (±0.093) | (±0.047) | (±0.025) | (±0.346) | (±0.925) | (±0.843) |
| [¹¹¹Indium-NTA] | | | | | | | | | | |
| DSPE:DSPC | − | 0.457 | − | − | 0.846 | − | − | 2.02 | − | − |
| (0.100) | | (±0.109) | − | − | (±0.079) | − | − | (±0.357) | − | − |
| DSPE:DSPC | + | 1.18 | − | − | 2.37 | − | − | 2.02 | − | − |
| (5:95) | | (±0.030) | − | − | (±0.363) | − | − | (±0.296) | − | − |
| DSPE:DSPC | + | 1.55 | − | − | 3.11 | − | − | 1.98 | − | − |
| (20:80) | | (±0.355) | − | − | (±0.833) | − | − | (±0.070) | − | − |
| DSPE:DSPC | − | 0.621 | − | − | 0.865 | − | − | 1.39 | − | − |
| (20:80)^{#} | | (±0.110) | − | − | (±0.150) | − | − | (±0.026) | − | − |
| DSPE:DSPC | + | 2.11 | − | − | 3.98 | − | − | 1.88 | − | − |
| (40:60) | | (±0.061) | − | − | (±0.512) | − | − | (±0.305) | − | − |
| DSPE:DSPC | + | 3.18 | − | − | 5.66 | − | − | 1.76 | − | − |
| (60:40) | | (±0.681) | − | − | (±1.48) | − | − | (±0.145) | − | − |
| DSPE:DSPC | + | 3.20 | − | − | 5.85 | − | − | 1.83 | − | − |
| (100:0) | | (±0.084) | − | − | (±0.803) | − | − | (±0.252) | − | − |

^{#}= PEGylation as per Examples 1 and 2
¹= % injected dose per gram of tissue, mean of 3 values ± SEM, except for * = mean of 4 values ± SEM.
²= tumour to blood ratio, mean of 3 values ± SEM, except for * = mean of 4 values ± SEM.
@ = historic control

TABLE 3
COMPARATIVE EXAMPLES OF THE INFLUENCE OF PEG
MODIFICATION ON TUMOUR LOCALISATION

| | | | |
|---|---|---|---|
| | | | Tumour to |
| PEG | Blood | Tumour | Blood |

| | Reference | mol % | 24 h | 48 h | 24 h | 48 h | 24 h | 48 h |
|---|---|---|---|---|---|---|---|---|
| DSPE:DSPC:CHOL | [1] | 0 | 7.6⁺ | 1.2⁺ | 3.9⁺ | 1.7⁺ | 0.5 | 1.4 |
| (1:10:3 mol ratio) | | | | | | | | |
| DSPE:DSPC:CHOL | | 7.14 | 15.1⁺ | 5.5⁺ | 4.2⁺ | 3.5⁺ | 0.28 | 0.64 |
| (1:10:3 mol ratio) | | | | | | | | |
| DSPE:HSPC:CHOL | [1] | 5.00 | 118* | 84* | 23* | 29.1* | 0.19 | 0.35 |
| (0.15:1.85:1 mol ratio) | | | | | | | | |
| DSPE:HSPC:CHOL | [2] | 6.25 | ˜105* | ˜90* | ˜0.5* | ˜2.2** | 0.0048 | 0.024 |
| (0.2:2:1) | | | | | | | | |
| DSPC:CHOL | [3] | 0 | − | 1.0 | − | 2.0 | − | 2.0 |
| (2:1) | | | | | | | | |
| DSPE:DSPC:CHOL | | 6.25 | − | 5.0 | − | 3.0 | − | 0.6 |
| (0.2:2:1) | | | | | | | | |

Table 3 - Key
⁺= % injected dose per gram of tissue
*= ug of doxorubicin/ml. of plasma or per ml of ascites tumour and fluid
**= ug of doxorubicin per g ascites cells
˜ = Approximations from graphs
[1] PCT/US90/66211.
[2] Papahadjopolos.
[3] Hwang et al.

### Example 5

### Lack of Correlation Between Exclusion of Liposomes PEGylated to Different Extents from the Liver/Spleen and Their Improved Tumour Concentration

The rationale for the LTI PCT/US 90/06211 PEG-liposome design is that, since the liver and spleen are the major organs of elimination and PEG compromises the uptake of liposomes by these organs, the ensuing retention of the liposomes in the blood will drive more liposomally entrapped compound into the tumour. When using PEG in this way, one simple corollary is that the improvement in tumour localisation (as opposed to entrapment or other mechanisms with impact on tumour drug concentrations) should be inversely related to liver and/or spleen localisation. In contrast if PEG has other effects such as improving retention in the tumour, the amount of PEG optimal for exclusion from the liver and spleen will not necessarily be identical to that giving the best localisation in the tumour.

FIG. 2 compares the effect of different mole percent of PEGylated DSPE in DSPE-DSPC liposomes (prepared by exterior PEGylation as in Example 1), on liver, spleen and tumour concentrations of ¹¹¹Indium at 24 h. It is evident from the results that, whereas maximum exclusion from liver and spleen is observed at 40 mol % PEGylated DSPE, the tumour concentration improved progressively. Note specifically that whereas with the liver the maximum changes lie between 5-20 and 20-40% PEGylated DSPE, and that with the spleen almost all change occurs between 20 and 40%, with the tumour there is a relatively regular progression for all changes in % PEGylated DSPE, at least up to 60%. FIG. 3 shows the poor correlation between liver and tumor uptake and FIG. 3 shows the poor correlation between spleen and tumour uptake. The FIGS. 3a and 3b show results for individual mice and confirm the results of FIG. 2. Most of the improvement in tumour concentration (e.g. between 3 to 8.5% injected dose per gram tumour) occurs without any reciprocal change in liver or spleen concentration; these are both circa 15% injected dose per gram for the majority of tumour values over the above range. Thus some factor other than exclusion from the RES of the liver and spleen must account for the increment in tumour concentration of liposomal contents above circa 3% injected dose per gram.

This result shows that the factors influencing the reduction in liver and spleen uptake are not equivalent in all respects to those responsible for the improved tumour concentration of liposomally delivered compound (in this example ¹¹¹Indium chelated to NTA).

### Example 6

### Poor Predictive Value of 1 H and 24 H Latency in Selecting Liposomes for Maximised Tumour Dose of Entrapped Contents

In addition to reducing hapatosplenic uptake, polymers such as polyethylene glycol are also known to reduce adherence of serum proteins (cf. PCT/GB 89/01262) and this is associated with increased latency in serum (i.e. improved retention of contents) in some reports but not others (see below). Since increased latency would be associated with improved circulation times, when optimlsing for tumour localisation based on the “push” principle embodied in PCT/US 90/06211 one would select a liposome with high latency.

FIG. 4a-c shows the latency of liposomes containing ¹¹¹Indium chelated to NTA. Irrespective of the level of PEGylated DSPE present in the liposomes (1a and 1b), DSPE:DSPC liposomes with 0 to 100 mol % PEGylated DSPE showed negligible loss of contents when stored for at least 19 days in 50 mM phosphate 250 mM sucrose pH 7.4 at 4° C. Free ¹¹¹Indium (and excess TMPEG) was removed from loaded liposomes before storage using a Sepharose CL-4B 20 ml syringe barrel column. After storage for the times indicated, leakage of liposomally entrapped ¹¹¹Indium was assessed using paper chromatography of Whatman 4 filter paper and appropriate buffer (depending on liposome pH), run for approximately 8 cm. The upper and lower sections were counted for ¹¹¹In. The latency was corrected for free counts at time zero. Similar results were obtained with unPEGylated liposomes after 22 days storage (FIG. 4c). Storage of an ¹¹¹Indium loaded PEGylated liposome preparation for 49d without removal of the TMPEG and buffer (50 mM borate 250 mM sucrose pH 9.3) also resulted in a similar moderate loss of contents (FIG. 4d) to that seen with PEGylated liposomes from which TMPEG was removed prior to storage.

When these liposomes were exposed to citrated fresh frozen plasma for 1-24 h, there was a clear relationship between the amount of PEGylated DSPE and loss of latency (FIGS. 5a and 5b). Surprisingly, there was an approximately inverse relationship between latency after 24 h plasma exposure and tumour concentration of ¹¹¹Indium at 24 h (FIG. 6a). Although released In/NTA complexes can be anticipated to transfer the Indium to blood proteins and hence evade the RES capture that is characteristic of liposomes, injection of In/NTA complexes equivalent to the total liposomal contents resulted in blood levels at 24 h that were greater than those seen with 20% PEGylated DSPE but less than that seen with higher degrees of DSPE substitution (FIG. 6b). Similarly with the liver, the In/NTA gave lower liver uptake than all liposomal samples (FIG. 6b). Thus despite the marked and similar loss of latency in vitro at 24 h for the liposomes containing 40-100% PEGylated DSPE, the effect of liposomal encapsulation was still prominent. In addition, similar results with respect to enhanced tumour concentration and tumour to blood ratios >1 were obtained with different liposomal contents (e.g. ¹²⁵I-TI) that are known to be rapidly cleared when lost from liposomes (see FIG. 18 discussed in Example 9 below).

Since this inverse correlation is unexpected, possible artifactual sources of the discrepancy were sought: first whether a difference between mouse and human plasma could explain the anomaly, i.e. was mouse plasma a less potent stimulus of leakage (FIG. 7) and secondly whether complement or other heat labile factors in the human plasma might be responsible (FIG. 8). FIG. 7 compares the latency result with citrated human plasma (from FIG. 5a and b) to the latency for liposomes exposed to heparinised mouse plasma for 1 and 24/25 h. Although mouse plasma led to less loss of latency at 24/25 h, particularly evident at 40-100% PEGylated DSPE, the mouse plasma results, like the human plasma results are still showing an inverse relationship with tumour ¹¹¹Indium concentration and are thus indicating that plasma induced loss of latency is not a reliable predictor of PEG-liposome behaviour with respect to tumour drug concentration.

FIG. 8 compares the latency result with citrated human plasma (from FIG. 5a and b) to the latency for liposomes exposed to citrated human plaza which had been heat treated at 56° C. for 45 min to remove complement and other heat labile lipid transfer factors (previously reported). As with mouse plasma, there was some difference after 24 h incubation, evident with 40-100% PEGylated DSPE liposomes, but there is still an inverse relationship with tumour ¹¹¹Indium concentration.

One possible explanation for failure of 1 and 24/25 h latency estimates to serve as good predictors of tumour drug concentration might be that distribution to the tumour largely occurs before the first hour of circulation. If loss of latency were relatively slow with respect to the rate of influx of the liposomes into the tumour, late loss of latency might have little impact and hence explain the discrepancy observed above. The temporal relationship between latency and plasma exposure was therefore explored using liposomes with 20 mol % PEGylated DSPE. FIG. 9 shows the impact of 2-60 min exposure to fresh frozen citrated plasma. At only 2 minutes, the ¹¹¹Indium liberated was 42% of the original entrapped material and this only rose to 53% after 1 h implying that most of the plasma induced loss occurs in ≦52 min. Thus, even if latency is measured at very early time points similar results are obtained to the 1 h measurement. Inspection of FIG. 18a (below) shows that distribution to the tumour in <2 min could not account for the tumour drug concentration benefit.

Whole blood has also been reported to have a less marked effect on latency than plasma and this could potentially explain an in vivo/in vitro discrepancy. However, using PEGylated DSPE containing liposomes (20 mol %) equivalent to 20 μg of lipid, incubated with 300 μl heparinised mouse blood at 37° C. for 1 h, 56.4% of the entrapped Indium was released. In comparable 1 h exposures to plasma 22.8% ¹¹¹In was released with human citrated fresh frozen plasma, 55.9% with mouse plasma and 43.6% with heat inactivated human plasma, thus plasma/blood differences are unlikely to explain the in vivo/in vitro discrepancy.

In order to ascertain the basis of the serum/plasma induced loss of latency (i.e. whether it is due to lipid composition or PEGylation), the latency of unPEGylated and PEGylated liposomes was compared. FIG. 10 compares the PEGylated and DSPE containing liposomes with their unPEGylated counterparts (the latency of the 0% DSPE liposomes is indicated by the dashed line). There was a concentration dependent effect of DSPE on latency, evident after 1 and 24 h incubation. At all concentrations of DSPE, the loss of latency was not markedly different in unPEGylated and PEGylated examples, demonstrating that PEG had little impact on the loss of latency. This result is similar to previous findings but at variance with others [Blume and Cevc, (1993)] who showed an effect from circa 2% PEG-PE (using incorporated PEG-lipid).

Despite the surprising inverse relationship between plasma-induced loss of latency and tumour ¹¹¹In concentration, there was evidence that some loss of liposomal contents may have been occurring in vivo, since in contrast to the other normal organs where the PEGylated DSPE content correlated with either exclusion (liver and spleen) or modest enhancement of vain content (heart, lung, colon, muscle skin). The kidney showed more markedly enhanced ¹¹¹In content. This enhanced renal ¹¹¹In content approximately correlated to the amount of ¹¹¹Indium released by mouse plasma (FIG. 11). The filled lines show the regressions and dotted curves the 95% confidence intervals. However, injected free ¹¹¹ln/NTA complexes, equivalent to the total liposome contents, produced lower renal ¹¹¹In levels at 24 h than either PEGylated or unPEGylated liposomes containing 60 and 100% DSPE (FIG. 12).

Since leakage of ¹¹¹Indium was thus apparently occurring in vivo, one concern might be that the inverse correlation observed between latency and tumour localisation could be due to loss of ¹¹¹Indium, subsequent binding to plasma proteins and the latters' tendency to extavasate into tumours. However, whereas the % injected dose per gram of tumour increased progressively in PEGylated liposomes containing 20%, 60% and 100% DSPE (see FIG. 1) the equivalent doses of free ¹¹¹Indium/NTA (i.e. the dose available for leakage) gave only 57.5% of the tumour concentration for the 100% DSPE liposomes and 82.0% for the 60% DSPE liposomes, but 137% of the 20% DSPE liposomes (which had a much higher latency than the first two preparations). Thus leakage of ¹¹¹Indium cannot explain the inverse correlation observed.

Since rapid loss of latency is intrinsically undesirable for many liposomal formulations, changes in lipid composition may be sought to remedy this. However, it is important to appreciate, that optimisation should still be driven by tumour concentration and tumour:blood or tumour to tissue ratios. Several changes in lipid formulation are known to have beneficial impact on latency in serum/plasma and incorporation of cholesterol is widely used. However, this does not necessarily lead to improved tumour concentrations (cf. the example in Table 2. utilising PEGylated liposomes containing DSPE:DSPC:cholesterol 5:62:33 mol % compared with the PEGylated DSPE:DSPC 5:95 mol % of FIG. 1 which gave 1.70±0.27 and 2.37±0.36 injected dose per gram of tumour at circa 24 h post injection respectively).

### Example 7

### Influence of PEGylated DSPE Content on Biodistribution in Normal Tissues Other Than Liver, Spleen and Kidney

Biodistribution studies were performed using DSPE:DSPC liposomes prepared as described in Example 1. Distribution profiles were compared with the blood levels (all concentrations given are at 24 h). FIG. 13 shows the tissue concentrations (left panels) and tissue:blood ratios. The tissue concentrations were not corrected for the blood content of the organs. The concentrations of ¹¹¹Indium at 24 h rose in all organs with PEGylated liposomes containing increasing amounts of DSPE (left hand panels). In all organs there was a trend of decreasing tissue:blood ratios (right hand panels) up to 20 mol % DSPE, implying that the increase in blood levels was not accompanied by a parallel increase in tissue levels, hence that the PEG-lipid was slightly reducing entry into these tissues as well as its effect on liver and spleen (or that the “push” principle is predominating in these normal tissues). PEGylated liposomes with 40 or more mol % either maintained this reduced tissue:blood ratio (e.g. lung) or showed a slight increase, but never in excess of the unPEGylated 0% DSPE control.

### Example 8

### Indium Loading and Loss After the PEGylation Reaction and Buffer Exchange

When ¹¹¹Indium loading was performed before PEGylation, the DSPE content had no significance impact on the amount of ¹¹¹In entrapped (FIG. 14). Employing the reaction procedure using 166 mg/ml TMPEG in borate buffer at pH 9.3 and then exchanging the buffer prior to injection/use, it was noted that there was substantial loss of ¹¹¹In which varied depending on the DSPE concentration (FIG. 15). Area under the curve estimates indicate that the PEGylated DSPE has a complex effect, improving then worsening ¹¹¹In loss (FIG. 16). However, this large loss of latency did not occur during PEGylation since the liposome, TMPEG, borate buffer reaction mixture still retained over 80% of its ¹¹¹In after 49d storage (see FIG. 4d). Thus the loss of ¹¹¹In appears to be due to the pH change from 9.3 to 7.4 when the buffer is exchanged. To confirm this, unPEGylated liposomes formed at pH 9.3 were exposed to a buffer exchange and retained only 33.9, 2.6 and 1.7% of their contents for 20, 60 and 100 mol % DSPE liposomes respectively (note that this ¹¹¹In loss is in excess of the equivalent PEGylated examples which retained 38.4, 9.3 and 7.4% respectively). This differential sensitivity to pH-change induced lysis is potentially important with respect to estimation of the extent of PEGylation since, if there is any heterogeneity of PEGylation, the more heavily PEGylated liposomes are more likely to resist loss of contents particularly at high DSPE levels. The pH change induced leakage could be based on lipid vesiculation (where lipid buds off the bilyayer on the side with the higher pH). Formation of hexagonal II structures also cannot be excluded because although DSPE forms hexagonal II at about 80° C., this temperature decreases with reduced hydration (from PEG) and with lowering of pH PEG-DSPE would be unable to form Hexagonal II (the 5 nm core would have difficulty accommodating the bulky PEG headgroup). With either scenario, PEG-lipid estimates based on than layer chromatography may be underestimates of the PEGylation status of the contents-bearing liposomes, whose behaviour is monitored in vivo.

The selection of pH 9.3 in Example 1 was based on

1) previous observations of enhanced PEGylation rates;

2) The benefits of generating a net negative charge of the phosphatidylethanolamine and hence tending to reduce the possibility of PEG-induced aggregation of the liposomes.

However aggregation could be reduced by using step-wise addition of low amounts of TMPEG and a more protracted PEGylation period can be used to overcome the delay in PEGylation at lower pH.

### Example 9

### Tumour and Skin Show Higher Increments in Tissue to Blood Ratios Than Other Organs

Using PEGylated DOPC:DOPE liposomes with 21 mol % DOPE, only a modest prolongation of circulation time was observed (FIG. 17). Comparison of the plots of ¹²⁵I-tyraminylinulin concentration versus time and tissue to blood ratios (FIGS. 18a and b), showed that skin and tumour show the highest increments of tissue to blood ratios with respect to unPEGylated controls. Significantly, given that some anti-tumour agents are cardiotoxic, there was no significant change in heart:blood ratios. Calculation of AUC's for 1-144 h (FIG. 19a) showed that whereas with unpEGylated liposomes the AUC for the tumour was less than that for kidney, skin and lung, after PEGylation only the kidney AUC₁₋₁₄₄ was in excess of the tumour (and the kidney:tumour ratio was reduced from 3.16:1 to 1.49:1 FIG. 19b).

DSPE:DSPC liposomes also showed a similar relationship between the increments in tumour to blood ratios and skin to blood ratios and showed that the effect was related to the concentration of PEGylated DSPE in the liposomes. In this experiment, in order to offset the impact that PEG has on circulation time (and hence allow the effect of PEG concentration on other features of biodistribution to predominate), the PEGylated liposomes with only 5% DSPE contained 33 mol % cholesterol and 62% DSPC whereas the PEGylated liposomes with 40 mol % DSPE contained only DSPC. This expedient produced blood elimination curves that were similar over the 22-72 h time period. FIG. 20a and b compares PEGylated liposomes with 5 mol % DSPE plus cholesterol to those with 40 mol % DSPE. The additional PEG-DSPE content causes an increase in the slope of tumour to blood ratio and skin to blood ratio, but does not increase the ratios in other organs and decreases them in liver and spleen. The apparent increase in muscle and decrease in lung ratios was not a consistent finding (see FIG. 18a and b). These results show that even with minimal changes in blood clearance rates (due to the compensating presence of cholesterol in the less heavily PEGylated liposomes), PEG still has an impact on tumour to blood and skin to blood ratios. This provides further evidence that the “push” and “trap” principles operate independently.

Table 4 shows the blood and skin results and skin to blood ratios for a range of different liposomal formulations.

These findings indicate some similarities in the behaviour of tumour tissues and skin with respect to the PEG liposomes of this invention. Thus the liposomal preparation should have utility in delivery of compounds selectively to skin.

TABLE 4
THE INFLUENCE OF PEG MODIFICATION ON SKIN LOCALISATION

| composition of | | Blood¹ | | | Skin¹ | | | Skin to Blood² | | |
|---|---|---|---|---|---|---|---|---|---|---|
| lipid (mol %) | PEG | 22-25 h | 60-70 h | 110-147 h | 22-25 h | 60-70 h | 110-147 h | 22-25 h | 60-70 h | 110-147 h |
| (contents) | # | (±SEM) | (±SEM) | (±SEM) | (±SEM) | (±SEM) | (±SEM) | (±SEM) | (±SEM) | (±SEM) |
| DSPE:DSPC:Chol | + | 0.66 | 0.29 | 0.095 | 1.01 | 1.08 | 1.87 | 1.89 | 4.43 | 19.7 ± |
| (5:62:33) | | (±0.134) | (±0.101) | (±0.004) | (±0.160) | (±0.249) | (±0.423) | (±0.123) | (±1.09) | (4.60) |
| (¹¹¹In-NTA) | | | | | | | | | | |
| DSPE:DSPC:Chol | − | 1.26 | 0.33 | 0.21 | 0.47 | 1.911 | 3.62 | 1.98 | 5.95 | 16.704 |
| (5:62:33) | | (±0.057) | (±0.052) | (±0.029) | (±0.601) | (±0.083 | (±0.978) | (±0.520) | (±0.687) | (±2.63) |
| (¹¹¹In-NTA) | | | | | | | | | | |
| DSPE:DSPC | + | 1.90 | 0.42 | − | 3.30 | 3.05 | − | 1.89 | 7.51 | − |
| (40:60) | | (±0.646) | (±0.107) | − | (±3.40) | (±0.414) | − | (±0.419) | (±1.04) | − |
| (¹¹¹In-NTA) | | | | | | | | | | |
| DOPE:DOPC | + | 0.38* | 0.19 | 0.063* | 1.01* | 1.21 | 0.629* | 2.90* | 6.166 | 9.80* |
| (21:79) | | (±0.044) | (±0.021) | (±0.007) | (±0.124) | (±0.323) | (±0.211) | (±0.721) | (±1.01) | (±2.50) |
| (¹¹¹I- | | | | | | | | | | |
| Tyraminylinulin) | | | | | | | | | | |
| DOPE:DOPC | − | 0.25 | 0.065 | 0.041* | 0.503 | 0.222 | (0.092* | 1.99 | 3.57 | 2.77* |
| (21:79) | | (±0.0283) | (±0.005) | (±0.009) | (±0.125) | (±0.029) | (±0.024) | (±0.264) | (±0.780) | (±1.13) |
| (¹¹¹I- | | | | | | | | | | |
| Tyraminylinulin) | | | | | | | | | | |
| DSPE-DSPC | − | 0.457 | − | − | 0.709 | − | − | 1.68 | − | − |
| (0:100) | | (±0.018) | − | − | (±0.108) | − | − | (±0.304) | − | − |
| (¹¹¹In-NTA) | | | | | | | | | | |
| DSPE:DSPC | + | 1.18 | − | − | 1.65 | − | − | 1.39 | − | − |
| (5:95) | | (±0.0383) | − | − | (±0.269) | − | − | (±0.190) | − | − |
| (¹¹¹In-NTA) | | | | | | | | | | |
| DSPE:DSPC | + | 1.55 | − | − | 1.65 | − | − | 1.15 | − | − |
| (20:80) | | (±0.355) | − | − | (±0.072) | − | − | (±0.187) | − | − |
| (¹¹¹In-NTA) | | | | | | | | | | |
| DSPE:DSPC | − | 0.621 | − | − | 0.878 | − | − | 1.39 | − | − |
| (20:80)* | | (±0.110) | − | − | (±0.195) | − | − | (±0.083) | − | − |
| (¹¹¹In-NTA) | | | | | | | | | | |
| DSPE:DSPC | + | 2.11 | − | − | 2.56 | − | − | 1.21 | − | − |
| (40:60) | | (±0.062) | − | − | (±0.180) | − | − | (±0.062) | − | − |
| (¹¹¹In-NTA) | | | | | | | | | | |
| DSPE:DSPC | + | 3.18 | − | − | 4.35 | − | − | 1.41 | − | − |
| (60:40) | | (±0.6806) | − | − | (±0.95) | − | − | (±0.18) | − | − |
| (¹¹¹In-NTA) | | | | | | | | | | |
| 1DSPE:DSPC | − | 2.11 | − | − | 3.69 | − | − | 1.76 | − | − |
| (60:40)* | | (±0.128) | − | − | (±0.350) | − | − | (±0.169) | − | − |
| (¹¹¹In-NTA) | | | | | | | | | | |
| DSPE:DSPC | + | 3.20 | − | − | 4.93 | − | − | 1.55 | − | − |
| (100:0) | | (±0.084) | − | − | (±0.719) | − | − | (±0.242) | − | − |
| (¹¹¹In-NTA) | | | | | | | | | | |
| DSPE:DSPC | − | 1.28 | − | − | 5.92 | − | − | 1.82 | − | − |
| (100:0)* | | (±0.312) | − | − | (±0.389) | − | − | (±0.096) | − | − |
| (¹¹¹In-NTA) | | | | | | | | | | |
| DOPE:DOPC | + | 0.732 | − | 0.122** | 3.11 | − | 4.77** | 4.57 | − | 39.36** |
| (21:79) | | (±0.147) | − | | (±0.144) | − | | (±0.879) | − | |
| (¹¹¹In-NTA) | | | | | | | | | | |
| DOPE:DOPC | − | 1.16 | − | 0.137** | 2.75 | − | 2.43** | 2.43 | − | 18.17** |
| (21:79) | | (±0.230) | − | | (±0.499) | − | | (±0.419) | − | |
| (¹¹¹In-NTA) | | | | | | | | | | |

^{#}= PEGylation as per examples 1 and 2
¹= % injected dose per gram of skin, mean of 3 values ± SEM, except for * = mean of 4 values ± SEM and for ** = mean of 2 values.
²= skin to blood ratio, mean of 3 values ± SEM except for * = mean of 4 values ± SEM and for ** = mean of 2 values.
@ = historic control

### Example 10

### The Influence of Liposome Lipid Composition on Tumour to Blood Ratio

In the prior art, when optimising for the “push” principle, not only has PEG been added to extend the plasma half-life, but also the lipid composition has been selected with that objective. The liposomes of this invention have not been optsmised in that way, but with respect to enhanced tumour entrapment (as revealed by an increased concentration within the tumour, accompanied by increased or maintained tumour to blood ratios with respect to controls). Also, it should be noted from the model-derived analysis of tumour to blood ratios given above, that, where the underlying liposomal formulation achieves good tumour entrapment in its own right (i.e. before the addition of PEG) then adequate PEGylation will further enhance both tumour concentrations and tumour to blood ratios. Only in situations where the underlying liposomes have inferior tumour entrapment properties and the PEGylation is insufficient to elicit a significant improvement in tumour entrapment, will the tumour to blood ratio fall as the tumour concentration of liposomes increases.

FIG. 21 shows the effect of different lipid compositions on blood and tumour levels at circa 24 h post injection. It is evident that there are significant differences in the tumour and blood concentrations for the different lipid compositions (A=DSPE:DSPC:CHOL 5:62:33 mol %; B=DOPE:DOPC 21:79 mol %; C=DSPC; D=DSPE:DSPC 20:80 mol %; E=DSPE:DSPC 60:40 mol %; F=DSPE). It should be noted that all tumour to blood ratios are higher than unity.

The type of contents within the liposome also has a bearing on the results. The compositions E and F had markedly reduced latency on exposure to plasma (see Example 6). These preparations (and others, A, C and D in FIG. 21) contained ¹¹¹Indium chelated to NTA. NTA is a relatively weak chelator and leakage of contents is known to result in transfer of leaked ¹¹¹Indium from NTA to plasma proteins (e.g. albumin). This conveys to the ¹¹¹Indium a relatively long circulation time and since albumin is known to be able to extravasate into tumours, this effect will complicate the analysis of tumour to blood ratios. Since E and F lost 93.2% and 91.9% (see FIG. 10) of their contents with 24 h exposure to plasma in vitro, the result for an amount of free NTA-¹¹¹Indium (filled bar) equivalent to the liposome contents is given for comparison. Note that the leakage of ¹¹¹Indium (shown above in FIG. 9 to occur mainly within the first 2 min of exposure to plasma) cannot completely account for the blood levels, i.e. that liposomal tumour entrapment has increased 24 h levels despite extremely low in vitro latency result of preparation F.

One important corollary of this observation is that, with contents with this type of behaviour, loss of latency may be less important than with contents which are rapidly eliminated after leakage from the liposome. It should also be noted by comparison to Table 2 (although not compared within the same experiment and thus not allowing statistical analysis of the significance of differences), that where the contents were NTA-¹¹¹Indium the impact of PEGylation appeared less marked for composition E and was possibly lost for composition F (where in both cases loss of latency was very high) as opposed to composition D where the impact of PEG was marked and loss of latency was less pronounced. If genuine, this apparent reduction in the impact of PEGylation is presumably due to the influence of leaked ¹¹¹Indium and its tendency to adhere to albumin.

### Example 11

### Estimation of Hydrolysis Rates of TMPEG and Other Competing Side Reactions by ¹⁹F-NMR

During PEGylation reactions it is important to monitor the rate of hydrolysis of the TMPEG and other competing side reactions. If the duration of the reaction time required for full PEGylation is protracted, e.g. ≧2 h, then this is a significant problem since TMPEG hydrolysis in aqueous solutions occurs relatively rapidly. FIG. 22 shows the ¹⁹F-nmr of TMPEG (the preparation used in Examples 1 and 2 in dimethyl sulphoxide (d6). The two triplets were observed, centered at −60.7752 ppm and −60.8856 ppm. The former corresponds to the ester linked tresyl group and the latter to the free trifluorethane sulphonic acid (TFESA). The TFESA represented 20% of the total sample. hydrolysis is likely to depend on pH, therefore each buffer system used must be assessed independently. On exposure to

1) 50 mM borate buffer pH 9.3 containing sucrose 250 mM;

2) 20 mM HEPES, 145 mM NaCl, pH 7.4, hydrolysis, there was progressive loss of the triplet for the ester-linked tresyl group (FIG. 23).

However, species other that TFESA were formed. In addition to the two triplets at −62.5 ppm (TMPEG) and −63.5 ppm (TFESA), which have shifted values from equivalent DMSO preparations, there were also peaks at circa −75 ppm and −120 ppm (FIG. 24). The nature of the species producing the peak at −75 ppm is unknown, but it appears to be a minor contaminant in the preparation which appeared to remain unchanged on exposure to buffer. By contrast the species at −63.4 and −120 ppm altered with time. Integrals of signals normalised to the −75 ppm signal, showed that the specie at −62.5 ppm decreased with time whilst that at −120 ppm increased; the specie at −63.5 ppm, the free TFESA, was relatively unchanging. The changes occurred much faster at pH 9.3 (FIG. 25a) than at pH 7.3 (FIG. 25b).

The most likely explanation for the −120 ppm specie is that it is free fluoride ions released by a side reaction of the TMPEG.

The inference of this result is that TMPEG is lost relatively rapidly, thus reaction mixtures may need to have serial aliquots of fresh TMPEG added to obtain high levels of PEGylation. At low pH the following reaction predominates:

—CH₂OSO₂CH₂CF₃+R—NH₃→—CH₂NH—R+HOSO₂CH₂CF₃

whereas at high pH the alternative reaction (which generates a —O—SO₂— coupling moiety) is predominant:

—CH₂OSO₂CH₂CF₃+R—NH₂+3 NaOH→—CH₂OSO₂NH—R+CH₃COOH+3NaF+H₂O

### Example 12

### PEGylation of DOPC:DOPE Liposomes Produces Increased Tumour to Blood Ratios for Delivery of Both ¹²⁵I-Labelled TI and ¹¹¹In

Given the difference in the behaviour after leakage of different liposomal contents it may be important to investigate the same lipid formulation with different contents. Two extreme examples are tyraminylinulin (TI) and ¹¹¹Indium chelated to NTA. As outlined above the former is small and rapidly removed via renal excretion once leaked from the liposome. In contrast, ¹¹¹Indium is transferred from NTA to plasma or extravascular proteins after liposome disruption. In order to assess how this affects our selection criteria, these two types of liposomal contents were assessed in the same liposomes.

The blood pharmacokinetics, tumour biodistribution and tumour to blood concentration ratios were analysed for ¹²⁵-labelled TI and ¹¹¹In loaded into control and PEGylated DOPC:DOPE (79:21 mol %) liposomes. The preparation of control and PEGylated DOPC:DOPE liposomes loaded with ¹²⁵I-labelled TI liposomes has been described in Example 2.

The preparation of control and PEGylated DOPC:DOPE liposomes loaded with ¹¹¹In was as follows: DOPC:DOPE (79:21 mol %) containing ionophore A23817 (1.08 mg per mg of lipid) were prepared by extrusion of the lipid suspension (10 mg/ml) in Hepes 20 mM pH 7.4, sodium chloride 145 mM and NTA 1 mM (the lipid suspension was obtained by vortexing followed by several cycles of warming up to 65° C. for 2 min and vortexing for 1 min and then subjected to 5 cycles of freezing and thawing and subsequent extrusion as in Example 2). The buffer was subsequently exchanged to Hepes 20 mM pH 7.4, sodium chloride 145 mM using a PD-10 column. For ¹¹¹In loading, 1.2 ml of liposomes at 3 mg/ml were incubated with 0.3 mCi of ¹¹¹In for 30 min at 65° C. ¹¹¹In loaded liposomes were isolated by GPC in a PD-10 column. The incorporation of ¹¹¹Indium to the liposomes was circa 100% as shown by paper chromatography as described in Example 6. The extruded liposomes loaded with ¹¹¹In were then PEGylated by reaction with TMPEG for 2 h at room temperature in Hepes 20 mM pH 7.4 containing sodium chloride 145 mM as described in Example 2.

FIG. 26 shows the blood pharmacokinetics and tumour biodistribution for ¹¹¹In and ¹²⁵I-labeleld TI entrapped in DOPC:DOPE ((79:21) mol %) liposomes. Blood levels for ¹¹¹In were slightly greater than blood levels for ¹²⁵I-labelled TI at all time points. The tumour biodistribution was very different for the two contents: while levels of ¹¹¹In were raising with time post-injection until they reached a plateau, levels of ¹²⁵I-labelled TI decreased with time post-injection. This behaviour is consistent with leakage of at least one of the contents from the liposomal vesicle.

Table 5 shows the blood and tumour concentrations and the tumour to blood concentration ratios at 24 h post-injection for ¹¹¹In and ¹²⁵I-labelled TI entrapped in control and PEGylated DOPC:DOPE lipsomes. For both contents, the tumour to blood concentration ratios were greater with the PEGylated liposomes than with the control liposomes (indicating that the “trapping” principle was operating). The type of contents did, however, influence the absolute value of tumour to blood ratios, but both contents exhibited a similar proportional increment in tumour to blood ratios. Thus with either of the contents used here that test criteria for demonstrating the “trapping” of liposomes were little affected.

Given the lower levels in the tumour with the tyraminylinulin, this provides a more rigorous test for the liposomal fate and is the preferred evaluation method. However, if the contents to be delivered have similar behaviour to ¹¹¹In, the contents actually to be delivered via the liposomes in question are more relevant.

TABLE 5
THE INFLUENCE OF CONTENTS ON TUMOUR ENTRAPMENT
BY PEG-MODIFICATION

| | | | | Tumour |
|---|---|---|---|---|
| Lipid | | | | to |
| composition | | Blood¹ | Tumour¹ | blood |
| (mol %) | | 22-25 h | 22-25 h | 22-25 h |
| [contents] | PEG# | (±SEM) | (±SEM) | (±SEM) |
| DOPC:DOPE | + | 0.73 | 3.40 | 5.04 |
| (79:21) | | (±0.15) | (±0.40) | (±1.08) |
| [¹¹¹Indium-NTA] | | | | |
| DOPC:DOPE | − | 1.16 | 2.96 | 2.59 |
| (79:21) | | (±0.23) | (±0.53) | (±0.3) |
| [¹¹¹Indium-NTA] | | | | |
| DOPC:DOPE | + | 0.38* | 1.39* | 3.89* |
| (79:21) | | (±0.04) | (±0.16) | (±0.75) |
| [¹¹¹Indium- | | | | |
| Tyraminylinulin] | | | | |
| DOPC:DOPE | − | 0.25 | 0.47 | 1.93 |
| (79:21) | | (±0.03) | (±0.09) | (±0.35) |
| [¹²⁵I- | | | | |
| Tyraminylinulin] | | | | |

# = PEGylation as per Example 2
1 = % injected dose per gram of tissue mean of 3 values ± SEM, except for * = mean of 4 values ± SEM.

1. A composition of a diagnostically or therapeutically effective agent for administration via the bloodstream to a solid tumour or the skin, the composition comprising a lipid-containing multi-molecular structure, the agent being present predominantly in the lipid-containing multi-molecular structure, wherein the lipid-containing multi-molecular structure comprises one or more hydrophobic entities bearing covalently bound hydrophilic polymer moieties, and wherein the physical form of the lipid-containing multi-molecular structure, the nature of the hydrophobic entities, the nature of the hydrophilic polymer moieties, the ratio of the polymer-bearing hydrophobic entities to non-derivatised hydrophobic entities exposed to the bloodstream and, when there are two or more hydrophobic entities, the relative proportions of the hydrophobic entities, are all selected such that:
(i) on intravenous injection of the composition to an animal, where appropriate bearing a model solid tumour, the ratio of tumour concentration to blood concentration or the ratio of skin concentration to blood concentration of the agent achieved at either or both of 24 and 48 hours following the injection is greater than unity,
(ii) the ratio of tumour to blood concentrations or the ratio of skin concentration to blood concentration of the agent achieved at 24 and 48 hours following intravenous injection of the composition to an animal, where appropriate bearing a model solid tumour, is not significantly lower than the ratio of tumour concentration to blood concentration or than the ratio of skin concentration to blood concentration of the agent achieved at the same times after intravenous injection to an animal, where appropriate bearing a model solid tumour, of a first control product, which is identical to the composition except that the first control product lacks any hydrophilic polymer modification of the hydrophobic entities, and
(iii) except in the case where the composition consists essentially of an agent associated with a lipid-containing multi-molecular structure consisting of one or more species of hydrophobic entity, each specie being susceptible to derivatisation with hydrophilic polymer moieties and where at least a portion of each of the species of hydrophobic entities is derivatised with hydrophilic moieties, the tumour concentration or skin concentration of the agent achieved by intravenous injection of the composition to an animal, where appropriate bearing a model solid tumour, is greater at 24 and 48 hours following injection than is the tumour concentration or skin concentration of the agent achieved by intravenous injection to an animal of a second control product, which is identical to the composition except that the second control product lacks any hydrophilic polymer modification and lacks any hydrophobic entities which are derivatlsed by polymer modification in the composition, or, in the case where the composition consists essentially of an agent associated with a lipid-containing multi-molecular structure which consists of one or more species of hydrophobic entity, each specie being susceptible to derivatisation with hydrophilic polymer moieties and where at least a portion of each of the species of hydrophobic entities is derivatised with hydrophilic moieties, the tumour concentration or skin concentration of the agent achieved by intravenous injection of the composition to an animal, where appropriate bearing a model solid tumour, is greater at 24 and 48 hours following injection than is the tumour concentration or skin concentration of the agent achieved by intravenous injection to an animal of the first control product as defined above.

2. A composition according to claim 1 wherein the lipid-containing multi-molecular structure comprises liposomes.

3. A composition according to claim 1 or claim 2 wherein the covalently bound hydrophilic polymer moieties are polyethylene glycol moieties.

4. A composition according to claim 3 wherein the diagnostically or therapeutically effective agent is entrapped within liposomes bearing polyethylene glycol moieties covalently linked to phosphatidylethanolamine molecules at least on the external surface of the liposomes.

5. A composition according to claim 4 wherein the polyethylene glycol moieties are linked by a non-biodegradable covalent bond obtainable by treating phosphatidyl ethanolamine or liposomes containing phosphatidyl ethanolamine with a derivative of 2,2,2-trifluoroethane sulphonyl polyethylene glycol.

6. A composition according to claim 5 wherein the derivative is the monomethyl ether of 2,2,2-trifluoroethane sulphonyl polyethylene glycol.

7. A composition according to any one of claims 1 to 6 wherein the hydrophilic polymer is a polyethylene glycol having a molecular weight of from 250 to 12000.

8. A composition according to any preceding claim wherein the diagnostically or therapeutically effective agent is an agent for diagnosing or treating dermatological diseases or disorders.

9. A composition according to any preceding claim wherein the diagnostically or therapeutically effective agent is an agent for diagnosing or treating solid tumours.

10. A composition according to claim 9 wherein the agent is a tumour imaging agent.

11. A composition according to claim 9 wherein the agent is a cytostatic or cytotoxic agent.

12. A composition according to any one of the preceding claims wherein lipid-containing multi-molecular structure comprises liposomes containing at least half of the total amount of the diagnostic or therapeutic agent in the composition.

13. A composition according to any preceding claim wherein the ratio of tumour concentration to blood concentration or of skin concentration to blood concentration of the agent achieved at 24 to 48 hours is greater than the ratio of tumour concentration to blood concentration or of skin concentration to blood concentration of the agent achieved at the same times by the first control product.

14. A composition according to claim 13 wherein the tumour concentration or skin concentration of the agent remains greater than the blood concentration achieved by administration of the composition throughout the period from 24 to 48 hours after administration.

15. A composition according to any preceding claim for use in a method of diagnosis or therapy practised on the human or animal body.

16. Use of a composition according to any preceding claim in the production of a medicament for use in the diagnosis or treatment of deratological diseases or disorders or solid tumours in the human or animal body.

17. A method of treating or diagnosing a dermatological disease or disorder or a solid tumour in a human or animal patient which method comprises administering an effective non-toxic amount of a composition according to any preceding claim to said patient.

18. A method according to claim 17 comprising a further step of systemic or localised treatment of said skin or said tumour to secure delivery of the diagnostic or therapeutic agent.

19. A method according to claim 18 wherein said further step comprises local application of heat or local, or systemic administration of an agent which disrupts lipid-containing multi-molecular structures so as to render said structure leaky or fusogenic.

20. A process for producing a composition of a diagnostically or therapeutically effective agent for administration via the bloodstream to a solid tumour or the skin, the composition comprising a lipid-containing multi-molecular structure, the agent being present predominantly in the lipid-containing multi-molecular structure, wherein the lipid-containing multi-molecular structure comprises one or more hydrophobic entities bearing covalently bound hydrophilic polymer moieties, which process comprises selecting the physical form of the lipid-containing multi-molecular structure, the nature of the hydrophobic entities, the nature of the hydrophilic polymer moieties, the ratio of the polymer-bearing hydrophobic entities to non-derivatised hydrophobic entities exposed to the bloodstream and, when there are two or more hydrophobic entities, the relative proportions of the hydrophobic entities, such that:
(i) on intravenous injection of the composition to an animal, where appropriate bearing a model solid tumour, the ratio of tumour concentration to blood concentration or the ratio of skin concentration to blood concentration of the agent achieved at either or both of 24 and 48 hours following the injection is greater than unity,
(ii) the ratio of tumour to blood concentrations or the ratio of skin concentration to blood concentration of the agent achieved at 24 and 48 hours following intravenous injection of the composition to an animal, where appropriate bearing a model solid tumour, is not significantly lower than the ratio of tumour concentration to blood concentration or than the ratio of skin concentration to blood concentration of the agent achieved at the same times after intravenous injection to an animal, where appropriate bearing a model solid tumour, of a first control product, which is identical to the composition except that the first control product lacks any hydrophilic polymer modification of the hydrophobic entities, and
(iii) except in the case where the composition consists essentially of an agent associated with a lipid-containing multi-molecular structure consisting of one or more species of hydrophobic entity, each specie being susceptible to derivatisation with hydrophilic polymer moieties and where at least a portion of each of the species of hydrophobic entities is derivatised with hydrophilic moieties, the tumour concentration or skin concentration of the agent achieved by intravenous injection of the composition to an animal, where appropriate bearing a model solid tumour, is greater at 24 and 48 hours following injection than is the tumour concentration or skin concentration of the agent achieved by intravenous injection to an animal of a second control product, which is identical to the composition except that the second control product lacks any hydrophilic polymer modification and lacks any hydrophobic entities which are derivatised by polymer modification in the composition, or, in the case where the composition consists essentially of an agent associated with a lipid-containing multi-molecular structure which consists of one or more species of hydrophobic entity, each specie being susceptible to derivatisation with hydrophilic polymer moieties and where at least a portion of each of the species of hydrophobic entities is derivatised with hydrophilic moieties, the tumour concentration or skin concentration of the agent achieved by intravenous injection of the composition to an animal, where appropriate bearing a model solid tumour, is greater at 24 and 48 hours following injection than is the tumour concentration or skin concentration of the agent achieved by intravenous injection to an animal of the first control product as defined above.

21. A process according to claim 20 for producing a composition according to any one of claims 2 to 15.
